(19) United States
(12) Patent Application Publication
(10) Pub. No.: US 2013/0157258 A1
(43) Pub. Date: Jun. 20, 2013

(54) HCV NS5B PROTEASE MUTANTS

(71) Applicants: Douglas J. Bartels, North Liberty, IA (US); Min Jiang, Lexington, MA (US); Tara Lynn Kieffer, Brookline, MA (US); Olivier Nicolas, Montreal (CA)

(72) Inventors: Douglas J. Bartels, North Liberty, IA (US); Min Jiang, Lexington, MA (US); Tara Lynn Kieffer, Brookline, MA (US); Olivier Nicolas, Montreal (CA)

(73) Assignee: VERTEX PHARMACEUTICALS INCORPORATED, Cambridge, MA (US)

(21) Appl. No.: 13/704,107

(22) PCT Filed: Jun. 15, 2011
(86) PCT No.: PCT/US11/40568

§ 371 Date: Mar. 1, 2013

Related U.S. Application Data
(60) Provisional application No. 61/357,850, filed on Jun. 23, 2010.
(60) Provisional application No. 61/355,014, filed on Jun. 15, 2010.

(51) Int. Cl. C12Q 1/70

(52) U.S. Cl. CPC C12Q 1/707
USPC 435/5; 435/194; 435/188; 530/329; 530/328; 530/327; 530/326; 530/325; 530/324; 530/387.9; 536/23.2; 435/320.1; 435/252.33; 435/325; 435/358; 435/346; 435/254.2; 435/254.21; 435/419; 435/414; 435/412; 435/415

(57) ABSTRACT

The invention provides polypeptides comprising an amino acid sequence comprising at least one variation from wild-type HCV NS5B polymerase, the at least one variation selected from the group consisting of cysteine, isoleucine, valine, or proline at amino acid position 419; alanine, valine, or asparagine at amino acid position 482; valine, isoleucine, threonine, or serine at amino acid position 486; and isoleucine at amino acid position 494, as the amino acid positions are defined in SEQ ID NO: 1, and having Hepatitis C Virus (HCV) NS5B polymerase activity. Polynucleotides encoding the polypeptide, antibodies, host cells, compositions, and methods for detecting an HCV NS5B polymerase having resistance to a polymerase inhibitor also are provided.

### CROSS-REFERENCE TO RELATED APPLICATIONS

This application claims priority to U.S. Provisional Patent Application No. 61/357,850, filed Jun. 23, 2010, and U.S. Provisional Patent Application No. 61/355,014, filed Jun. 15, 2010, the disclosures of which are incorporated by reference in their entirety.

### TECHNICAL FIELD OF THE INVENTION

The invention generally relates to Hepatitis C virus NS5B polymerase mutants and uses thereof.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: ASCII (text) file named “45408B_SeqListing.txt,” 2,592,060 bytes, created on Jun. 14, 2011.

### BACKGROUND OF THE INVENTION

Infection by hepatitis C virus (“HCV”) is a compelling human medical problem. HCV is recognized as the causative agent for most cases of non-A, non-B hepatitis, with an estimated human sero-prevalence of 3% globally (Alberti et al., J. Hepatology, 31 (Suppl. 1), 17-24 (1999)). Nearly four million individuals may be infected in the United States alone (Alter et al, Gastroenterol. Clin. North Am., 23, 437-455 (1994); Alter, J. Hepatology, 31 (Suppl. 1), 88-91 (1999)).

Upon first exposure to HCV, only about 20% of infected individuals develop acute clinical hepatitis; others appear not to develop significant outward symptoms of infection. In almost 70% of instances, however, the virus establishes a chronic infection that persists for decades (Iwarson, FEMS Microbiology Reviews, 14, 201-204 (1994); Lavanchy, J. Viral Hepatitis, 6, 35-47 (1999)). This usually results in recurrent and progressively worsening liver inflammation, which often leads to more severe disease states such as cirrhosis and hepatocellular carcinoma (Kew, FEMS Microbiology Reviews, 14, 211-220 (1994); Saito et al., Proc. Natl. Acad. Sci. USA, 87, 6547-6549 (1990)). Unfortunately, there are no broadly effective treatments for the debilitating progression of chronic HCV.

HCV comprises a single-stranded positive-sense RNA genome encoding a polyprotein of 3010-3033 amino acids, which is co- or post-translationally processed into structural proteins (e.g., core, E1, and E2) and nonstructural (NS) proteins (e.g., NS2, NS3, NS4A, NS4B, NS5A, and NS5B) (Choo et al., Proc. Natl. Acad. Sci. USA, 88, 2451-2455 (1991); Kato et al., Proc. Natl. Acad. Sci. USA, 87, 9524-9528 (1990); Takamizawa et al., J. Virol., 65, 1105-1113 (1991); Choo et al., Science, 244, 359-362 (1989)). Host peptidase first cleaves the polyprotein to release the structural proteins (Hijikata et al., Proc. Natl. Acad. Sci. USA, 88, 5547-5551 (1991); Lin et al., J. Virol., 68, 5063-5073 (1994)). The NS2/3 metalloprotease cleaves at the NS2/NS3 junction. NS3 (with cofactor NS4A) displays serine protease activity and further processes the viral polyprotein to generate the majority of the viral enzymes essential for viral replication and infectivity, including NS4B, NS5A, and NS5B proteins (Bartenschlager et al., J. Virol., 67, 3835-3844 (1993)).

All nonstructural proteins play a role in HCV replication and/or packaging, and antiviral agents targeting of NS3 protease and NS5B polymerase have shown a great deal of promise in the clinic. NS5B is an RNA-dependent RNA polymerase (RdRp) and terminal transferase, and plays a key role in replication of the viral RNA genome (Lohmann et al., J. Virol., 71, 8416-8428 (1997); Lohmann et al., Virology, 249, 108-118 (1998); Kolykhalov et al., J. Virol., 74(4), 2046-2051 (2000)). The NS5B protein comprises approximately 591 amino acids (65 kDa) having canonical motifs common to other RNA viral polymerases.

The current standard of care for HCV infection, pegylated interferon alpha in combination with ribavirin, has roughly 40% sustained viral response (SVR) for patients infected with genotype 1, which counts for 70% of chronic hepatitis C patients in developed countries, and 80% SVR in genotype 2 or 3 HCV-infected patients (McHutchison et al., N. Engl. J. Med., 339, 1485-1492 (1998); Davis et al., N. Engl. J. Med., 339, 1493-1499 (1998); McHutchinson et al., N. Engl. J. Med., 361, 580-539 (2009)). Moreover, HCV is prone to developing resistance to antiviral drugs, further complicating development of successful therapeutic regimens.

Thus, there is a need for more effective anti-HCV therapies, particularly agents that inhibit HCV replication and methods of estimating if HCV therapies will be successful. An understanding of HCV resistance mutants would further progress towards effective HCV treatments.

### SUMMARY OF THE INVENTION

The invention provides materials and methods that are useful for identifying and improving antiviral therapeutics, including therapeutics for HCV infection; and materials and methods that are useful for diagnosing HCV infection and/or characterizing the strain, genotype, and/or phenotype of HCV, which is useful for selecting a treatment regimen. More specifically, the invention relates to nucleic acids that encode polypeptides with NS5B polymerase activity, and to polypeptides with NS5B polymerase activity, and polynucleotide and polypeptide fragments; to modified versions of each of the foregoing; and to methods of making and methods of using each of the foregoing. In some variations, the polypeptides and polynucleotides of the invention are isolated and/or purified. In some variations of the invention, the polypeptides and polynucleotides have non-naturally occurring modifications, such as attachment of a heterologous signal peptide, tag sequence, or fusion partner to a polypeptide (or attachment of a sequence encoding one or more of these to a polynucleotide); attachment of one or more labels; inclusion of a nucleotide or amino acid that does not naturally occur in HCV polynucleotides or polypeptides; attachment of heterologous expression control sequences to polynucleotides; and mixing in solvents, buffers, pharmaceutical delivery vehicles, or other compositions that are man-made. The invention further relates to antibody substances and that recognize NS5B, and cells that produce such antibodies, and antibody substances modified as described.

Wild-type NS5B amino acid and polynucleotide sequences are set forth in SEQ ID NO: 1 and SEQ ID NO: 2, respectively. These sequences are referred to herein in the description of the invention. For example, variations of the invention relate to polypeptides and polynucleotides that differ from SEQ ID NO: 1 and SEQ ID NO: 2 at one or more amino acids/codons, as described below in greater detail. The amino acid sequences set forth in SEQ ID NO: 3 and SEQ ID NO: 516 are defined to encompass exemplary polypeptides of the invention. SEQ ID NO: 1 and SEQ ID NO: 2 are NS5B polymerase sequences from HCV genotype 1b, and polypeptide and polynucleotides from other HCV genotypes or subtypes, e.g., HCV genotype 1a, are specifically contemplated as part of the invention.

In some variations, the invention provides an isolated polypeptide comprising Hepatitis C Virus (HCV) NS5B polymerase activity and comprising an amino acid sequence comprising at least one variation from SEQ ID NO: 1. The variation(s) are selected from the group consisting of cysteine, isoleucine, valine, or proline at amino acid position 419; alanine at amino acid position 423; alanine, threonine, valine, or asparagine at amino acid position 482; valine, isoleucine, threonine, or serine at amino acid position 486; and/or isoleucine at amino acid position 494, as the amino acid positions are defined in SEQ ID NO: 1. The amino acid of a polypeptide that corresponds to a position as defined in SEQ ID NO: 1 is identifiable by aligning the sequence of the polypeptide with the sequence of SEQ ID NO: 1 in a manner that maximizes sequence identity.

An isolated polypeptide comprising an amino acid sequence at least 90% identical to the amino acid sequence of SEQ ID NO: 3 or at least 90% identical to a portion of SEQ ID NO: 3 having Hepatitis C Virus (HCV) NS5B polymerase activity also is provided. At least one of the amino acids of the polypeptide that correspond to positions 419, 423, 482, 486, and/or 494 of SEQ ID NO: 3 is identical to the corresponding amino acid(s) of SEQ ID NO: 3. The invention further includes an isolated polypeptide comprising an amino acid sequence at least 90% identical to the amino acid sequence of SEQ ID NO: 516 or at least 90% identical to a portion of SEQ ID NO: 516 having Hepatitis C Virus (HCV) NS5B polymerase activity, with the proviso that at least one of the amino acids of the polypeptide that correspond to positions 419, 482, 486, and/or 494 of SEQ ID NO: 516 is identical to the corresponding amino acid(s) of SEQ ID NO: 516.

Additionally, the invention provides an isolated peptide having no more than 50 amino acids and comprising a sequence of 6-50 amino acids that is at least 90% identical to a portion of the amino acid sequence of SEQ ID NO: 3 encompassing at least one of amino acid residues 419, 423, 482, 486, and 494. When present, at least one of the amino acids of the polypeptide that corresponds to positions 419, 423, 482, 486, and/or 494 of SEQ ID NO: 3 is identical to the corresponding amino acid(s) at the same position of SEQ ID NO: 3. Also included is an isolated peptide having no more than 50 amino acids and comprising a sequence of 6-50 amino acids that is at least 90% identical to a portion of the amino acid sequence of SEQ ID NO: 516 encompassing at least one of amino acid residues 419, 482, 486, and 494, with the proviso that, when present, at least one of the amino acids of the polypeptide that corresponds to positions 419, 482, 486, and/or 494 of SEQ ID NO: 516 is identical to the corresponding amino acid(s) of SEQ ID NO: 516. In some instances, the polypeptide comprises the amino acid sequence of SEQ ID NOs: 4-502.

Isolated polynucleotides encoding the polypeptides are provided, such as an isolated polynucleotide comprising a nucleotide sequence encoding a polypeptide having Hepatitis C Virus (HCV) NS5B polymerase activity. The nucleotide sequence comprises at least one codon variation from SEQ ID NO: 2, the at least one codon variation from SEQ ID NO: 2 selected from the group consisting of a codon encoding cysteine, isoleucine, valine, or proline at codon 419; a codon encoding alanine at codon 423; a codon encoding alanine, threonine, valine, or asparagine at codon 482; a codon encoding valine, isoleucine, threonine, or serine at codon 486; and a codon encoding isoleucine at codon 494, as the codon positions are defined in SEQ ID NO: 2. In various aspects of the invention, the nucleotide sequence comprises a codon encoding a methionine at codon position 423 as defined in SEQ ID NO: 2. The invention further provides an isolated polynucleotide comprising no more than 50 nucleotides and comprising a nucleotide sequence of 14-50 nucleotides complementary to a continuous portion of the nucleotide sequence of the polynucleotide described herein, the portion including at least one codon at a codon position selected from codon positions 419, 423, 482, 486, and 494 of SEQ ID NO: 2.

The invention further includes a method of making a polypeptide of the invention, using a polynucleotide of the invention. For example, the invention includes growing a host cell transformed or transfected with a polynucleotide of the invention under conditions in which the cell expresses the encoded polypeptide. Optionally, the method further includes purifying the polypeptide from the cell or growth media of the cell.

The invention further encompasses a method for determining whether an HCV-infected patient is infected with an HCV strain that has a decreased sensitivity to VX-222, a method for characterizing the HCV inhibitory activity of an agent, a method for identifying an agent able to rescue the polymerase-inhibitor activity of VX-222 against an HCV NS5B polymerase having resistance to VX-222, and a method for detecting the presence of drug-resistant HCV in a sample comprising HCV.

The following numbered paragraphs each succinctly define one or more exemplary variations of the invention:

1. An isolated polypeptide comprising an amino acid sequence comprising at least one variation from SEQ ID NO: 1, the at least one variation selected from the group consisting of cysteine, isoleucine, valine, or proline at amino acid position 419; alanine at amino acid position 423; alanine, threonine, valine, or asparagine at amino acid position 482; valine, isoleucine, threonine, or serine at amino acid position 486; and isoleucine at amino acid position 494, as the amino acid positions are defined in SEQ ID NO: 1, wherein the polypeptide has Hepatitis C Virus (HCV) NS5B polymerase activity.

2. The isolated polypeptide of paragraph 1 comprising an amino acid sequence at least 75% identical to SEQ ID NO: 1.

3. The isolated polypeptide of paragraph 1 comprising an amino acid sequence at least 85% identical to SEQ ID NO: 1.

4. The isolated polypeptide of paragraph 1 comprising an amino acid sequence at least 90% identical to SEQ ID NO: 1.

5. The isolated polypeptide of paragraph 1 comprising an amino acid sequence at least 95% identical to SEQ ID NO: 1.

6. The isolated polypeptide of paragraph 1 comprising an amino acid sequence 100% identical to SEQ ID NO: 1 except for the at least one variation.

7. The isolated polypeptide of paragraph 1, wherein the amino acid sequence comprises at least 85% identity to a portion of SEQ ID NO: 1 having HCV NS5B polymerase activity.

8. The isolated polypeptide of any one of paragraphs 1-7, wherein the amino acid residue of the polypeptide corresponding to amino acid 419 of SEQ ID NO: 1 is cysteine, isoleucine, valine, or proline.

9. The isolated polypeptide of any one of paragraphs 1-7, wherein the amino acid residue of the polypeptide corresponding to amino acid 423 of SEQ ID NO: 1 is alanine.

10. The isolated polypeptide of any one of paragraphs 1-7, wherein the amino acid residue of the polypeptide corresponding to amino acid 482 of SEQ ID NO: 1 is alanine, threonine, valine, or asparagine.

11. The isolated polypeptide of any one of paragraphs 1-7, wherein the amino acid residue of the polypeptide corresponding to amino acid 486 of SEQ ID NO: 1 is valine, isoleucine, threonine, or serine.

12. The isolated polypeptide of any one of paragraphs 1-7, wherein the amino acid residue of the polypeptide corresponding to amino acid 494 of SEQ ID NO: 1 is isoleucine.

13. The isolated polypeptide of any one of paragraphs 9-12, wherein the amino acid residue of the polypeptide corresponding to amino acid 419 of SEQ ID NO: 1 is methionine or serine.

14. The isolated polypeptide of any one of paragraphs 8, 10, 11, and 12, wherein the amino acid residue of the polypeptide corresponding to amino acid 423 of SEQ ID NO: 1 is isoleucine, threonine, or valine.

15. The isolated polypeptide of any one of paragraphs 8, 9, 11, and 12, wherein the amino acid residue of the polypeptide corresponding to amino acid 482 of SEQ ID NO: 1 is leucine.

16. The isolated polypeptide of any one of paragraphs 8-12, wherein the amino acid residue of the polypeptide corresponding to amino acid 422 of SEQ ID NO: 1 is tyrosine.

17. The isolated polypeptide of paragraph 8, wherein the amino acid residue of the polypeptide corresponding to amino acid 423 of SEQ ID NO: 1 is alanine.

18. The isolated polypeptide of paragraph 8 or paragraph 17, wherein the amino acid residue of the polypeptide corresponding to amino acid 482 of SEQ ID NO: 1 is alanine, threonine, valine, or asparagine.

19. The isolated polypeptide of any one of paragraphs 8, 17, and 18, wherein the amino acid residue of the polypeptide corresponding to amino acid 486 of SEQ ID NO: 1 is valine, isoleucine, threonine, or serine.

20. The isolated polypeptide of any one of paragraphs 8 and 17-19, wherein the amino acid residue of the polypeptide corresponding to amino acid 494 of SEQ ID NO: 1 is isoleucine.

21. An isolated polypeptide comprising an amino acid sequence at least 90% identical to the amino acid sequence of SEQ ID NO: 3 or at least 90% identical to a portion of SEQ ID NO: 3 having Hepatitis C Virus (HCV) NS5B polymerase activity, with the proviso that at least one of the amino acids of the polypeptide that correspond to positions 419, 423, 482, 486, and/or 494 of SEQ ID NO: 3 is identical to the corresponding amino acid(s) of SEQ ID NO: 3.

22. The isolated polypeptide of paragraph 21, wherein the amino acid residue of the polypeptide corresponding to amino acid 419 of SEQ ID NO: 3 is cysteine, isoleucine, valine, or proline.

23. The isolated polypeptide of paragraph 21, wherein the amino acid residue of the polypeptide corresponding to amino acid 423 of SEQ ID NO: 3 is alanine.

24. The isolated polypeptide of paragraph 21, wherein the amino acid residue of the polypeptide corresponding to amino acid 482 of SEQ ID NO: 3 is alanine, threonine, valine, or asparagine.

25. The isolated polypeptide of paragraph 21, wherein the amino acid residue of the polypeptide corresponding to amino acid 486 of SEQ ID NO: 3 is valine, isoleucine, threonine, or serine.

26. The isolated polypeptide of paragraph 21, wherein the amino acid residue of the polypeptide corresponding to amino acid 494 of SEQ ID NO: 3 is isoleucine.

27. The isolated polypeptide of any one of paragraphs 23-26, wherein the amino acid residue of the polypeptide corresponding to amino acid 419 of SEQ ID NO: 3 is methionine or serine.

28. The isolated polypeptide of any one of paragraphs 22, 24, 25, and 26, wherein the amino acid residue of the polypeptide corresponding to amino acid 423 of SEQ ID NO: 3 is isoleucine, threonine, or valine.

29. The isolated polypeptide of any one of paragraphs 22, 23, 25, and 26, wherein the amino acid residue of the polypeptide corresponding to amino acid 482 of SEQ ID NO: 3 is leucine.

30. The isolated polypeptide of any one of paragraphs 22-26, wherein the amino acid residue of the polypeptide corresponding to amino acid 422 of SEQ ID NO: 3 is tyrosine.

31. The isolated polypeptide of paragraph 22, wherein the amino acid residue of the polypeptide corresponding to amino acid 423 of SEQ ID NO: 1 is alanine.

32. The isolated polypeptide of paragraph 22 or paragraph 31, wherein the amino acid residue of the polypeptide corresponding to amino acid 482 of SEQ ID NO: 1 is alanine, threonine, valine, or asparagine.

33. The isolated polypeptide of any one of paragraphs 22, 31, and 32, wherein the amino acid residue of the polypeptide corresponding to amino acid 486 of SEQ ID NO: 1 is valine, isoleucine, threonine, or serine.

34. The isolated polypeptide of any one of paragraphs 22 and 31-33, wherein the amino acid residue of the polypeptide corresponding to amino acid 494 of SEQ ID NO: 1 is isoleucine.

35. The isolated polypeptide of any one of paragraphs 1-35 attached to a detectable label. Exemplary labels include fluorescent dyes (e.g., fluorescein, Alexa, or green fluorescent protein), radioisotopes (e.g., ³⁵S, ¹²⁵I, ¹³¹I) or enzymes (e.g., horseradish peroxidase, alkaline phosphatase, secreted alkaline phophatase (SEAP), chloramphenicol acetyltransferase (CAT), luciferase, and β-galactosidase)

36. The isolated polypeptide of any one of paragraphs 1-35 attached to a heterologous peptide tag. Exemplary tags include a His tag, a FLAG tag, a hemaglutinin tag, a glutathione-S-transferase tag, a maltose binding protein tag, a green fluorescent protein tag, and a chitin binding protein tag. For these purposes, “heterologous” refers to a tag that does not naturally occur in HCV.

37. The isolated polypeptide of any one of paragraphs 1-35 fused to a heterologous peptide. For these purposes, “heterologous” refers to a peptide or polypeptide that does not naturally occur in HCV. Exemplary heterologous peptides include, e.g., an immunogenic peptide; a marker or reporter protein; a signal peptide; and fragments of any of the foregoing

38. The isolated polypeptide of any one of paragraphs 1-35 fused to a heterologous signal peptide. Heterologous signal peptides are signal peptides from non-HCV proteins, such as signal peptides from animal, plant, fungi, or other organisms. Preferred signal peptides include those for proteins naturally expressed by the cell or organism to be used to express the polypeptide of the invention.

39. An isolated peptide having no more than 50 amino acids and comprising a sequence of 6-50 amino acids that is at least 90% identical to a portion of the amino acid sequence of SEQ ID NO: 3 encompassing at least one of amino acid residues 419, 423, 482, 486, and 494, with the proviso that, when present, at least one of the amino acids of the polypeptide that corresponds to positions 419, 423, 482, 486, and/or 494 of SEQ ID NO: 3 is identical to the corresponding amino acid(s) of SEQ ID NO: 3.

40. The peptide of paragraph 39 comprising no more than 25 amino acids.

41. The peptide of paragraph 40 comprising no more than 15 amino acids.

42. The peptide of any one of paragraphs 39-41 capable of eliciting an antibody that specifically binds to a VX-222-resistant HCV NS5B polymerase.

43. An isolated polypeptide comprising the amino acid sequence of SEQ ID NOs: 4-502.

44. An isolated antibody or fragment thereof that specifically binds the polypeptide of any one of paragraphs 1-43.

45. The isolated antibody or fragment thereof of paragraph 44, that is an antibody fragment selected from the group consisting of an F(ab′)2, Fab, Fab′, Fv, Fc, or Fd fragment

46. The isolated antibody or fragment thereof of paragraph 44, wherein the antibody is a monoclonal antibody.

47. The isolated antibody or fragment thereof of paragraph 44 or paragraph 46, wherein the antibody is a chimeric antibody, a humanized antibody, or a human antibody.

48. A hybridoma that produces an antibody that specifically binds the polypeptide of any one of paragraphs 1-43.

49. An isolated polynucleotide comprising a nucleotide sequence encoding the polypeptide of any one of paragraphs 1-43.

50. An isolated polynucleotide comprising a nucleotide sequence encoding a polypeptide having Hepatitis C Virus (HCV) NS5B polymerase activity, the nucleotide sequence comprising at least one codon variation from SEQ ID NO: 2, the at least one codon variation from SEQ ID NO: 2 selected from the group consisting of a codon encoding cysteine, isoleucine, valine, or proline at codon 419; a codon encoding alanine at codon 423; a codon encoding alanine, threonine, valine, or asparagine at codon 482; a codon encoding valine, isoleucine, threonine, or serine at codon 486; and a codon encoding isoleucine at codon 494, as the codon positions are defined in SEQ ID NO: 2.

51. The isolated polynucleotide of paragraph 50, comprising a codon encoding cysteine, isoleucine, valine, or proline at the position in the nucleotide sequence corresponding to codon 419 of SEQ ID NO: 2.

52. The isolated polynucleotide of paragraph 50, comprising a codon encoding alanine at the position in the nucleotide sequence corresponding to codon 423 of SEQ ID NO: 2.

53. The isolated polynucleotide of paragraph 50, comprising a codon encoding alanine, threonine, valine, or asparagine at the position in the nucleotide sequence corresponding to codon 482 of SEQ ID NO: 2.

54. The isolated polynucleotide of paragraph 50, comprising a codon encoding valine, isoleucine, threonine, or serine at the position in the nucleotide sequence corresponding to codon 486 of SEQ ID NO: 2.

55. The isolated polynucleotide of paragraph 50, comprising a codon encoding isoleucine at the position in the nucleotide sequence corresponding to codon 494 of SEQ ID NO: 2.

56. An isolated polynucleotide comprising a nucleotide sequence that encodes at least one of the amino acid sequences selected from the group consisting of SEQ ID NOs: 4-502.

57. An isolated polynucleotide comprising no more than 50 nucleotides and comprising a nucleotide sequence of 14-50 nucleotides complementary to a continuous portion of the nucleotide sequence of the polynucleotide of paragraph 50, the portion including at least one codon at a codon position selected from codon positions 419, 423, 482, 486, and 494 of SEQ ID NO: 2.

58. The polynucleotide of paragraph 57 capable of sequence-specific hybridization to a polynucleotide comprising a nucleotide sequence encoding a VX-222-resistant HCV NS5B polymerase. Another variation of the invention is a kit containing 2, 3, 4, 5, 6, or more different polynucleotides of this type, for detecting mutants at two or more of these codons.

59. The polynucleotide of any one of paragraphs 49-58 further comprising a detectable label.

60. The polynucleotide of any one of paragraphs 49-59 operably linked to a heterologous expression control sequence.

61. A composition comprising the polynucleotide of any one of paragraphs 49-60 and a carrier.

62. An expression vector comprising the polynucleotide of any one of paragraphs 49-56 and 60. For purposes of this aspect of the invention, the term “expression vector: excludes HCV virus. An isolated HCV virus containing a polynucleotide of the invention is a separate and distinct aspect of the invention.

63. The expression vector of paragraph 62, wherein the expression vector is a viral vector.

64. The expression vector of paragraph 62 or paragraph 63, wherein the polynucleotide is operably linked to a heterologous expression control sequence.

65. The expression vector of any one of paragraphs 62-64 further comprising a 3′ non-translated region of an HCV genome.

66. An isolated cell comprising the expression vector of any one of paragraphs 62-65.

67. The isolated cell of paragraph 66, wherein the cell is a hepatocyte.

68. An isolated cell transformed or transfected with a polynucleotide according to any one of paragraphs 49-60, wherein the cell expresses a polypeptide encoding by the polynucleotide, and wherein the cell is free of HCV infection.

69. A hepatocyte cell line transformed or transfected with an HCV, wherein the nucleotide sequence of the HCV that encodes NS5B polymerase is identical to the nucleotide sequence of a polynucleotide of the invention.

70. A composition comprising the polypeptide of any one of paragraphs 1-43 and a carrier.

71. An assay mixture comprising an isolated polypeptide according to any one of paragraphs 1-43, an isolated RNA template, an RNA primer, nucleotide triphosphates, and buffer.

72. A method for determining whether an HCV-infected patient is infected with an HCV strain that has a decreased sensitivity to VX-222, the method comprising determining the presence or absence of the polypeptide of any one of paragraphs 1-34 in a biological sample from the patient, wherein the presence of the polypeptide indicates infection with an HCV strain that has a decreased sensitivity to VX-222. For the purposes of this method, “determining the presence or absence of the polypeptide of any one of paragraphs 1-34” should be understood to refer to evaluating the presence or absence of a polypeptide having an amino acid sequence identical to the sequence of the polypeptide defined in these paragraphs. The polypeptide in the sample may, for example, be part of an HCV polyprotein or in mixture with other proteins rather than an isolated polypeptide per se.

73. A method for characterizing the HCV inhibitory activity of an agent, the method comprising performing an HCV NS5B polymerase reaction with the polypeptide of any one of paragraphs 1-34 in the presence of an agent, and comparing polymerase activity in the presence of the agent with polymerase activity of the polypeptide in the absence of the agent.

74. The method of paragraph 73, wherein the method further comprises performing an HCV NS5B polymerase reaction in the absence of the agent.

75. The method of paragraph 73, wherein comparing polymerase activity comprises comparing the amount of polynucleotide generated by the NS5B polymerase reaction in the presence of the agent with the amount of polynucleotide generated by the NS5B polymerase reaction in the absence of the agent, wherein a decrease in the amount of polynucleotide generated by the polypeptide in the presence of the agent is indicative of HCV inhibitory activity.

76. A method for identifying an agent able to rescue the polymerase-inhibitor activity of VX-222 against an HCV NS5B polymerase having resistance to VX-222, the method comprising: a) performing an HCV NS5B polymerase reaction with the polypeptide of any one of paragraph 1-34 in the presence of an agent and VX-222; and b) comparing polymerase activity of the polypeptide in the presence of the agent with polymerase activity of the polypeptide in the absence of the agent, wherein a decrease in HCV polymerase activity in the presence of the agent is indicative of the ability to rescue the polymerase-inhibitory activity of VX-222 against an HCV NS5B polymerase having resistance to VX-222.

77. A method for determining whether an HCV-infected patient is infected with an HCV strain that has a decreased sensitivity to VX-222, the method comprising determining the presence or absence of the polynucleotide of any one of paragraphs 49-56 in a biological sample from the patient, wherein the presence of the polynucleotide indicates infection with an HCV strain having a decreased sensitivity to VX-222. For the purposes of this method, “determining the presence or absence of the polynucleotide of any one of paragraphs 49-56” should be understood to refer to evaluating the presence or absence of a polynucleotide having a nucleotide sequence identical to the sequence of the polynucleotide defined in these paragraphs. The polynucleotide in the sample may, for example, be an HCV genomic polynucleotide rather than an isolated polynucleotide per se.

78. A method for detecting the presence of drug-resistant HCV in a sample comprising HCV, wherein the method comprises determining the presence or absence of an HCV NS5B polypeptide with an amino acid sequence in which at least one amino acid that corresponds to positions 419, 423, 482, 486, and/or 494 of SEQ ID NO: 3 is identical to the corresponding amino acid(s) of SEQ ID NO: 3, wherein the presence of the at least one amino acid in the HCV NS5B protein indicates the presence of drug-resistant HCV.

79. The method of paragraph 72 or paragraph 78, wherein determining the presence or absence of the polypeptide comprises contacting the sample with an antibody or fragment thereof that specifically binds the polypeptide and detecting binding of the antibody or fragment thereof to the polypeptide.

80. A method for detecting the presence of drug-resistant HCV in a sample, wherein the method comprises determining the presence or absence of a polynucleotide in the sample, the polynucleotide comprising a nucleic acid sequence encoding HCV NS5B polymerase containing at least one codon selected from the group consisting of a codon encoding cysteine, isoleucine, valine, or proline at a position corresponding to codon position 419 of SEQ ID NO: 2; a codon encoding alanine at a position corresponding to codon position 423 of SEQ ID NO: 2; a codon encoding alanine, threonine, valine, or asparagine at a position corresponding to codon position 482 of SEQ ID NO: 2; a codon encoding valine, isoleucine, threonine, or serine at a position corresponding to codon position 486 of SEQ ID NO: 2; and a codon encoding isoleucine at a position corresponding to codon position 494 of SEQ ID NO: 2, wherein the presence of the at least one codon indicates the presence of drug-resistant HCV in the sample.

81. The method of paragraph 77 or paragraph 80, wherein the method comprises contacting polynucleotides in the sample with a nucleic acid probe that comprises a polynucleotide according to paragraph 58, under conditions allowing sequence-specific hybridization of the nucleic acid probe with a target sequence, wherein hybridization of the nucleic acid probe to a polynucleotide in the sample indicates the presence of drug-resistant HCV in the sample.

82. The method of paragraph 77 or paragraph 80, wherein the method comprises sequencing one or more polynucleotides in the sample to determine the presence the polynucleotide.

83. A method for determining whether an HCV-infected patient is infected with an HCV strain that has a decreased sensitivity to VX-222, the method comprising determining the presence or absence of a polypeptide in a biological sample from the patient, the polypeptide comprising an amino acid sequence comprising at least one variation from SEQ ID NO: 1, the at least one variation selected from the group consisting of cysteine, isoleucine, methionine, serine, valine, or proline at amino acid position 419; lysine at amino acid position 422; alanine, isoleucine, threonine, or valine at amino acid position 423; alanine, leucine, threonine, valine, or asparagine at amino acid position 482; valine, isoleucine, threonine, or serine at amino acid position 486; and isoleucine or alanine at amino acid position 494, as the amino acid positions are defined in SEQ ID NO: 1, wherein the presence of the polypeptide indicates infection with an HCV strain that has a decreased sensitivity to VX-222.

84. A method for identifying an agent able to rescue the polymerase-inhibitor activity of VX-222 against an HCV NS5B polymerase having resistance to VX-222, the method comprising: a) performing an HCV NS5B polymerase reaction with a polypeptide comprising an amino acid sequence comprising at least one variation from SEQ ID NO: 1, the at least one variation selected from the group consisting of cysteine, isoleucine, methionine, serine, valine, or proline at amino acid position 419; lysine at amino acid position 422; alanine, isoleucine, threonine, or valine at amino acid position 423; alanine, leucine, threonine, valine, or asparagine at amino acid position 482; valine, isoleucine, threonine, or serine at amino acid position 486; and isoleucine or alanine at amino acid position 494, as the amino acid positions are defined in SEQ ID NO: 1, in the presence of an agent and VX-222; and b) comparing polymerase activity of the polypeptide in the presence of the agent with polymerase activity of the polypeptide in the absence of the agent, wherein a decrease in HCV polymerase activity in the presence of the agent is indicative of the ability to rescue the polymerase-inhibitory activity of VX-222 against an HCV NS5B polymerase having resistance to VX-222.

85. The method of paragraph 83 or 84, wherein polypeptide comprises at least one variation selected from the group consisting of cysteine, isoleucine, valine, or proline at amino acid position 419; alanine, valine, or asparagine at amino acid position 482; valine, isoleucine, threonine, or serine at amino acid position 486; and isoleucine at amino acid position 494, as the amino acid positions are defined in SEQ ID NO: 1.

86. A method for determining whether an HCV-infected patient is infected with an HCV strain that has a decreased sensitivity to VX-222, the method comprising determining the presence or absence of a polynucleotide in a biological sample from the patient, the polynucleotide comprising a nucleic acid sequence encoding HCV NS5B polymerase containing at least one codon selected from the group consisting of a codon encoding cysteine, isoleucine, methionine, serine, valine, or proline at a position corresponding to codon position 419 of SEQ ID NO: 2; a codon encoding lysine at a position corresponding to codon position 422 of SEQ ID NO: 2; a codon encoding alanine, isoleucine, threonine, or valine at a position corresponding to codon position 423 of SEQ ID NO: 2; a codon encoding alanine, leucine, threonine, valine, or asparagine at a position corresponding to codon position 482 of SEQ ID NO: 2; a codon encoding valine, isoleucine, threonine, or serine at a position corresponding to codon position 486 of SEQ ID NO: 2; and a codon encoding isoleucine or alanine at a position corresponding to codon position 494 of SEQ ID NO: 2, wherein the presence of the polynucleotide indicates infection with an HCV strain having a decreased sensitivity to VX-222.

87. The method of paragraph 86, wherein polynucleotide comprises at least one codon selected from the group consisting of a codon encoding cysteine, isoleucine, valine, or proline at codon position 419 of SEQ ID NO: 2; a codon encoding alanine, valine, or asparagine at codon position 482 of SEQ ID NO: 2; a codon encoding valine, isoleucine, threonine, or serine at codon position 486 of SEQ ID NO: 2; and a codon encoding isoleucine at codon position 494 of SEQ ID NO: 2.

The foregoing summary is not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. Where embodiments concerning a polypeptide are described, embodiments involving polynucleotides that encode the polypeptide are specifically contemplated, and the reverse also is true. Where embodiments of the invention are described with respect to a specific NS5B polymerase mutant, it should be appreciated that analogous embodiments involving fragments, variants, analogs, and the like are specifically contemplated.

In addition to the foregoing, the invention includes, as an additional aspect, all embodiments of the invention narrower in scope in any way than the variations specifically mentioned above. With respect to aspects of the invention described as a genus, all individual species are individually considered separate aspects of the invention. For instance, if a polypeptide is described as having any one of two, three, four, or five amino acids at a specific position, then a polypeptide having each of the specific amino acids in that position is contemplated as an individual embodiment of the invention. Similarly, if a polypeptide is described in this manner with respect to two or more amino acid positions, then a polypeptide with each combination of amino acids is contemplated as a species of the invention. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated. If aspects of the invention are described as “comprising” a feature, embodiments also are contemplated “consisting of” or “consisting essentially of” the feature.

Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention. Additional features and variations of the invention will be apparent to those skilled in the art from the entirety of this application, and all such features are intended as aspects of the invention.

### DESCRIPTION OF THE FIGURES

FIGS. 1A-1C are a depiction of the nucleic acid sequence and the amino acid sequence encoding wild-type genotype 1b HCV NS5B polymerase, correlating the amino acid and codon positions within the sequences. The nucleic acid sequence is SEQ ID NO: 2 and the amino acid sequence is SEQ ID NO: 1 used herein.

FIGS. 2A and 2B are tables correlating the concentration of VX-222 and the frequency of resistant-colony formation of genotype 1a replicons and genotype 1b replicons.

FIGS. 3A-3C are an alignment of the amino acid sequence encoding wild-type genotype 1b HCV NS5B polymerase (SEQ ID NO: 1) and the amino acid sequence encoding wild-type genotype 1a HCV NS5B polymerase (SEQ ID NO: 514).

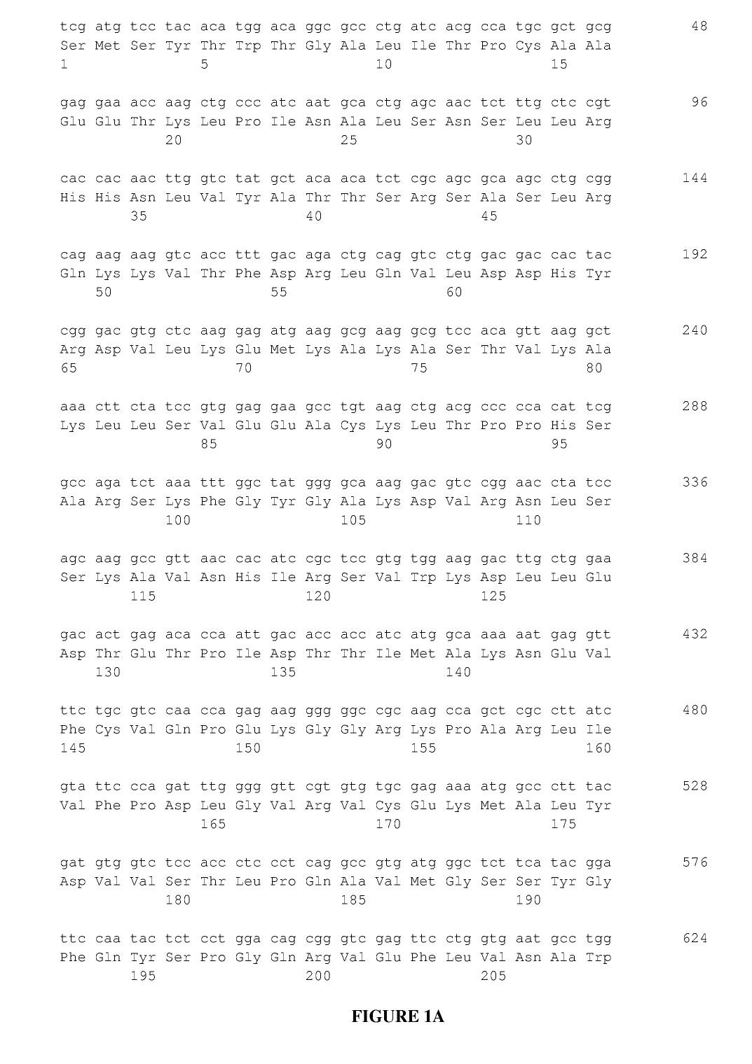

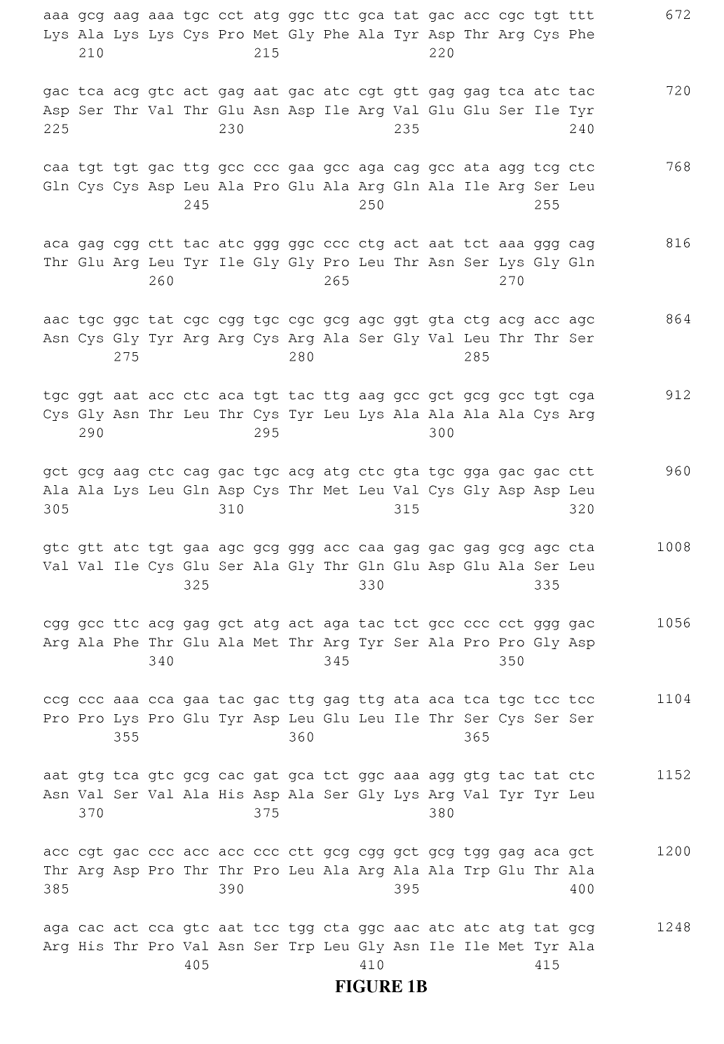

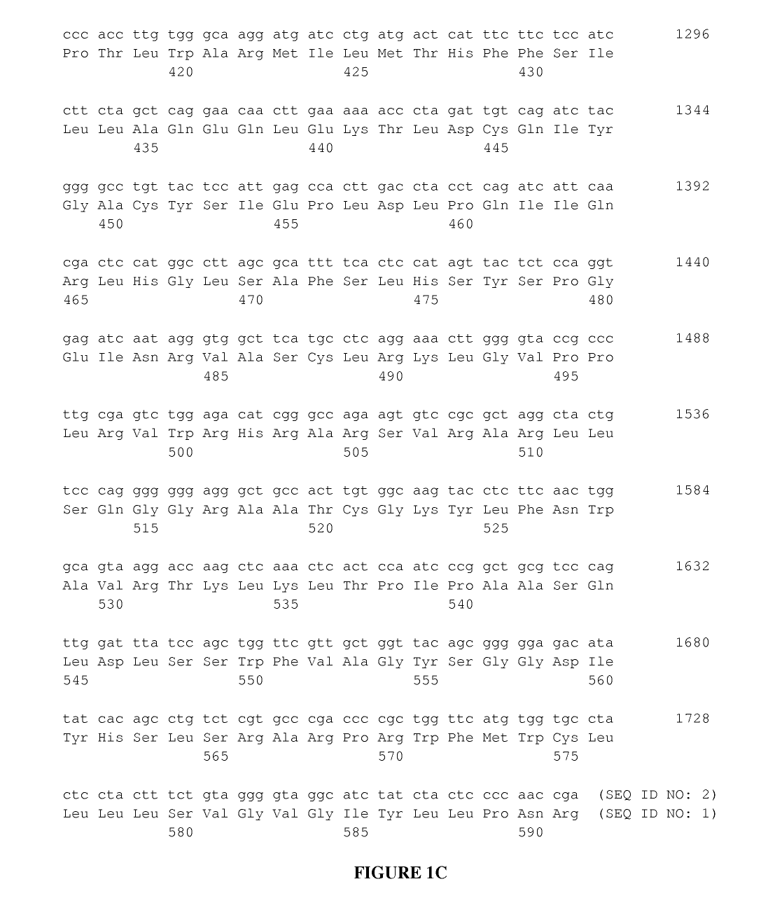

### DETAILED DESCRIPTION OF THE INVENTION

The invention is predicated, at least in part, on the surprising discovery of HCV strains containing particular mutations that render the HCV strains resistant to the therapeutic potential of HCV inhibitor compounds. In this regard, it has been determined that resistance to anti-viral agents, such as polymerase inhibitors, is accompanied by mutations in the Hepatitis C virus NS5B polymerase. These discoveries may be exploited in the design of therapies for the treatment of HCV infection.

### Polypeptides

The invention provides an isolated polypeptide comprising an amino acid sequence comprising at least one variation from SEQ ID NO: 1, the at least one variation selected from the group consisting of cysteine, isoleucine, valine, or proline at amino acid position 419; alanine at amino acid position 423; alanine, threonine, valine, or asparagine at amino acid position 482; valine, isoleucine, threonine, or serine at amino acid position 486; and isoleucine at amino acid position 494, wherein the polypeptide has HCV NS5B polymerase activity. The numbering system for the polypeptide used herein is in reference to the amino acid sequence of SEQ ID NO: 1, which is the amino acid sequence of a wild-type NS5B polymerase from HCV genotype 1b. The variations described herein are also contemplated for other genotype and subtype backgrounds, e.g., HCV genotype 1a. The amino acid sequence and nucleic acid sequence of wild-type HCV genotype 1a NS5B polymerase is set forth in SEQ ID NOs: 514 and 515, respectively. HCV NS5B polymerase polypeptides having the one or more described variations have been found to have resistance to at least one polymerase inhibitor, VX-222, described in, e.g., International Patent Publication Nos. WO 2008/058393 and WO 2002/100851, incorporated by reference in their entirety.

In one aspect, the polypeptide comprises a single variation at amino acid position 419, 423, 482, 486, or 496. Alternatively, the polypeptide comprises a single variation at amino acid position 419, 482, 486, or 494. Alternatively, the polypeptide comprises variations at two or more of amino acid positions 419, 423, 482, 486, and/or 496. All combinations of amino acids cysteine, isoleucine, valine, or proline at amino acid position 419; alanine at amino acid position 423; alanine, threonine, valine, or asparagine at amino acid position 482; valine, isoleucine, threonine, or serine at amino acid position 486; and/or isoleucine at amino acid position 494 are contemplated as variations characteristic of polypeptides of the invention. For example, the polypeptide comprising a cysteine, isoleucine, valine, or proline at amino acid position 419 also can comprise: an alanine at amino acid position 423 and/or an alanine, threonine, valine, or asparagine at amino acid position 482 and/or a valine, isoleucine, threonine, or serine at amino acid position 486 and/or isoleucine at amino acid position 494. Similarly, a polypeptide comprising an alanine at amino acid position 423 also can comprise: a cysteine, isoleucine, valine, or proline at amino acid position 419 and/or an alanine, threonine, valine, or asparagine at amino acid position 482 and/or a valine, isoleucine, threonine, or serine at amino acid position 486 and/or isoleucine at amino acid position 494. A polypeptide comprising an alanine, threonine, valine, or asparagine at amino acid position 482 also can comprise: a cysteine, isoleucine, valine, or proline at amino acid position 419 and/or an alanine at amino acid position 423 and/or a valine, isoleucine, threonine, or serine at amino acid position 486 and/or isoleucine at amino acid position 494. A polypeptide comprising a valine, isoleucine, threonine, or serine at amino acid position 486 also can comprise: cysteine, isoleucine, valine, or proline at amino acid position 419 and/or alanine at amino acid position 423 and/or alanine, threonine, valine, or asparagine at amino acid position 482 and/or isoleucine at amino acid position 494. A polypeptide comprising isoleucine at amino acid position 494 also can comprise: cysteine, isoleucine, valine, or proline at amino acid position 419 and/or alanine at amino acid position 423 and/or alanine, threonine, valine, or asparagine at amino acid position 482 and/or a valine, isoleucine, threonine, or serine at amino acid position 486.

In various embodiments, the polypeptide comprises a cysteine, isoleucine, valine, methionine, serine, or proline at amino acid position 419 and also comprises: an alanine, isoleucine, threonine, or valine at amino acid position 423 and/or an alanine, threonine, valine, leucine or asparagine at amino acid position 482 and/or a valine, isoleucine, threonine, or serine at amino acid position 486 and/or isoleucine or alanine at amino acid position 494. Alternatively, the polypeptide comprises an alanine, isoleucine, threonine, or valine at amino acid position 423 and further comprises: a cysteine, isoleucine, valine, methionine, serine, or proline at amino acid position 419 and/or an alanine, threonine, valine, leucine, or asparagine at amino acid position 482 and/or a valine, isoleucine, threonine, or serine at amino acid position 486 and/or isoleucine or alanine at amino acid position 494. A polypeptide comprising an alanine, threonine, valine, leucine, or asparagine at amino acid position 482 also comprises, in various aspects: a cysteine, isoleucine, valine, methionine, serine, or proline at amino acid position 419 and/or an alanine, isoleucine, threonine, or valine at amino acid position 423 and/or a valine, isoleucine, threonine, or serine at amino acid position 486 and/or isoleucine or alanine at amino acid position 494. A polypeptide comprising a valine, isoleucine, threonine, or serine at amino acid position 486 also can comprise: cysteine, isoleucine, valine, methionine, serine, or proline at amino acid position 419 and/or alanine, isoleucine, threonine, or valine at amino acid position 423 and/or alanine, threonine, valine, leucine, or asparagine at amino acid position 482 and/or isoleucine or alanine at amino acid position 494. A polypeptide comprising isoleucine or alanine at amino acid position 494 also can comprise: cysteine, isoleucine, valine, methionine, serine, or proline at amino acid position 419 and/or alanine, isoleucine, threonine, or valine at amino acid position 423 and/or alanine, threonine, valine, leucine, or asparagine at amino acid position 482 and/or a valine, isoleucine, threonine, or serine at amino acid position 486. Any of the polypeptides also comprises, in various embodiments, a lysine at amino acid position 422.

In one embodiment, the polypeptide comprising alanine at amino acid position 423; alanine, threonine, valine, or asparagine at amino acid position 482; valine, isoleucine, threonine, or serine at amino acid position 486; and/or isoleucine at amino acid position 494, also comprises a methionine or serine at position 419. Alternatively, the polypeptide comprises cysteine, isoleucine, valine, or proline at amino acid position 419; alanine, threonine, valine, or asparagine at amino acid position 482; valine, isoleucine, threonine, or serine at amino acid position 486; and/or isoleucine at amino acid position 494, and further comprises isoleucine, threonine, alanine, or valine at amino acid position 423. In another embodiment, the polypeptide comprises cysteine, isoleucine, valine, or proline at amino acid position 419; alanine at amino acid position 423; valine, isoleucine, threonine, or serine at amino acid position 486; and/or isoleucine at amino acid position 494, and further comprises leucine or threonine at amino acid position 482. In another embodiment, the polypeptide comprises cysteine, isoleucine, valine, or proline at amino acid position 419; alanine, threonine, valine, or asparagine at amino acid position 482; and/or valine, isoleucine, threonine, or serine at amino acid position 486, and further comprises alanine at amino acid position 494 (as well as, optionally, alanine at amino acid position 423). Any of the polypeptides may also include a lysine at amino acid position 422. The invention further provides an isolated polypeptide comprising (or consisting of) the amino acid sequence of any one of SEQ ID NOs: 4-502.

Optionally, the inventive polypeptide comprises additional variations with respect to the amino acid sequence of SEQ ID NO: 1 other than variations at positions 419, 423, 482, 486, and/or 494 due to natural deviation (e.g., differences in amino acid sequence among different HCV genotypes) or mutagenesis. HCV encompasses a heterogeneous family of virus having similar characteristics; for example, HCV is characterized as an enveloped RNA virus, approximately 50 nm in diameter, that primarily infects humans. Eleven HCV genotypes (numbered one through eleven) have been identified, many of which include more than one distinct subtype and multiple strains. The predominant HCV genotype worldwide is genotype 1, which encompasses two main subtypes, genotype 1a and genotype 1b. The different genotypes and subtypes share a common genomic structure (i.e., contain the same structural and non-structural genes), but differ with respect to genome sequences, pathogenicity, and prevalence in different areas of the world. While the NS5B polymerase is structurally and functionally similar among the different HCV genotypes, subtypes, and strains, the NS5B polymerase amino acid sequence may differ among different viral isolates (e.g., clinical viral isolates within the same genotype).

For example, NS5B polymerase polypeptides isolated from genotype 1b clinical isolates were sequenced, and the amino acid sequences were found to demonstrate a mean percent identity of about 92-96% compared to SEQ ID NO: 1 over 547 amino acids (N=538). The population of NS5B polymerases included polypeptides having amino acid sequences demonstrating about 87% identity to SEQ ID NO: 1. NS5B polymerase polypeptides isolated from genotype 1a clinical isolates were sequenced and found to comprise amino acid sequences demonstrating a mean percent identity of about 84-88% compared to SEQ ID NO: 1 over 547 amino acids, and the population included polymerases having sequences demonstrating about 76% identity to SEQ ID NO: 1 (N=239). The nucleic acid sequences of NS5B polymerase taken from genotype 1b and 1a clinical isolates were compared to the nucleic acid sequence of SEQ ID NO: 2 (i.e., the nucleic acid sequence of wild-type NS5B polymerase of HCV genotype 1b). The nucleic acid sequences encoding genotype 1b NS5B polymerase demonstrated a mean percent identity of about 92-94% to the nucleic acid sequence of SEQ ID NO: 2 over 1668 base pairs, and isolates were identified having 91% identity to SEQ ID NO: 2. Nucleic acid sequences encoding genotype 1a HCV NS5B polymerase demonstrated a mean percent identity of 80-82% to SEQ ID NO: 2, and isolates were identified having about 79% identity to SEQ ID NO: 2.

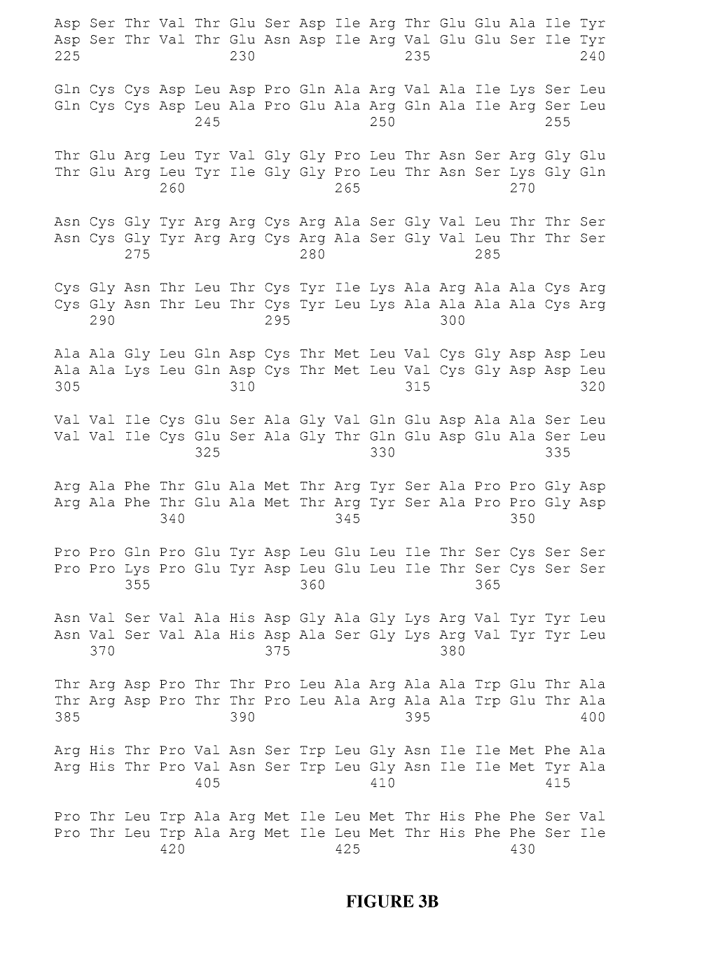

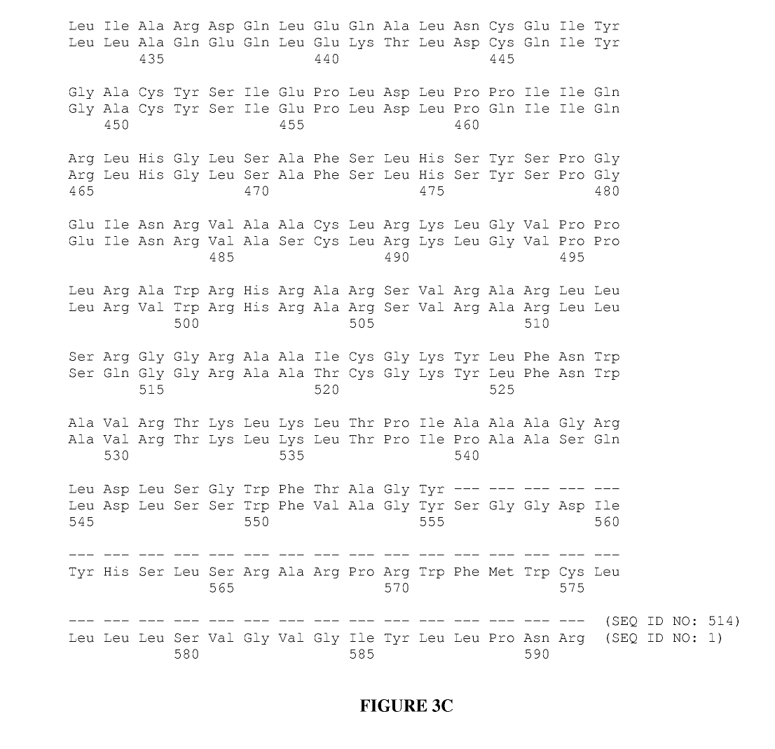

The amino acid and nucleic acid sequences of HCV NS5B polymerases from different HCV genotypes are disclosed in publicly available sequence databases, such as Genbank. Generally, the first four amino acids of the HCV NS5B polymerase sequence (SMSY (SEQ ID NO: 503)) are conserved among the different strains. While the amino acid residue positions described herein are in reference to SEQ ID NO: 1, the corresponding amino acids of other HCV NS5B polymerases (such as NS5B polymerases from HCV genotype 1a, genotype 2a, genotype 2b, genotype 2c, genotype 3a, or genotype 3b) can be identified by aligning the amino acid sequences with SEQ ID NO: 1. An exemplary alignment is provided in FIGS. 3A-3C, wherein the amino acid sequence of wild-type genotype 1b NS5B polymerase is aligned with the amino acid sequence of wild-type genotype 1a NS5B polymerase. Polypeptides having the variation(s) described herein relative to any of these other HCV genotypes are specifically contemplated as aspects of the invention.

In one aspect, the polypeptide (or a polynucleotide encoding a polypeptide) comprises an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the amino acid sequence of SEQ ID NO: 1 with the proviso that the amino acid sequence of the polypeptide comprises cysteine, isoleucine, valine, or proline at amino acid position 419; alanine at amino acid position 423; alanine, threonine, valine, or asparagine at amino acid position 482; valine, isoleucine, threonine, or serine at amino acid position 486; and/or isoleucine at amino acid position 494, as the amino acid positions are defined in SEQ ID NO: 1, and the polypeptide retains HCV NS5B polymerase activity. For example, in various embodiments, the polypeptide (or the polynucleotide encoding a polypeptide) comprises (or encodes) an amino acid sequence that is at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identical to the amino acid sequence of SEQ ID NO: 1 with the proviso that the amino acid sequence of the polypeptide comprises cysteine, isoleucine, valine, or proline at amino acid position 419; alanine, valine, or asparagine at amino acid position 482; valine, isoleucine, threonine, or serine at amino acid position 486; and/or isoleucine at amino acid position 494, as the amino acid positions are defined in SEQ ID NO: 1, and the polypeptide retains HCV NS5B polymerase activity.

In addition, the invention provides an isolated polypeptide comprising (or consisting of) an amino acid sequence at least 90% identical to the amino acid sequence of SEQ ID NO: 3 or at least 90% identical to a portion of SEQ ID NO: 3 having HCV NS5B polymerase activity, with the proviso that at least one of the amino acids of the polypeptide that correspond to positions 419, 423, 482, 486, and/or 494 of SEQ ID NO: 3 is identical to the corresponding amino acid(s) of SEQ ID NO: 3. In SEQ ID NO: 3, the amino acid residue at position 419 is cysteine, isoleucine, valine, or proline; the amino acid residue at position 423 is alanine; the amino acid residue at position 482 is alanine, threonine, valine, or asparagine; the amino acid residue at position 486 is valine, isoleucine, threonine, or serine; and the amino acid residue at position 494 is isoleucine. The invention further provides an isolated polypeptide comprising an amino acid sequence at least 90% identical to the amino acid sequence of SEQ ID NO: 516 or at least 90% identical to a portion of SEQ ID NO: 516 having Hepatitis C Virus (HCV) NS5B polymerase activity, with the proviso that at least one of the amino acids of the polypeptide that correspond to positions 419, 482, 486, and/or 494 of SEQ ID NO: 516 is identical to the corresponding amino acid(s) of SEQ ID NO: 516.

“Sequence identity” means that two amino acid or polynucleotide sequences are identical over a region of comparison, such as a region of at least about 250 residues of SEQ ID NO: 1. Optionally, the region of identity spans at least about 100-500 residues SEQ ID NO: 1 (e.g., the region of identity spans 150-400 residues of SEQ ID NO: 1, spans 200-350 residues of SEQ ID NO: 1, spans 250-300 residues of SEQ ID NO: 1, or spans over all 591 residues of SEQ ID NO: 1), and spans the active domain of the polypeptide. Several methods of conducting sequence alignment are known in the art and include, for example, the homology alignment algorithm (Needleman & Wunsch, J. Mol. Biol., 48, 443 (1970)); the local homology algorithm (Smith & Waterman, Adv. Appl. Math., 2, 482 (1981)); and the search for similarity method (Pearson & Lipman, Proc. Natl. Acad. Sci. USA, 85, 2444 (1988)). Preferably, the algorithm used to determine percent sequence identity and sequence similarity is the BLAST algorithm (Altschul et al., J. Mol. Biol., 215, 403-410 (1990); Henikoff & Henikoff. Proc. Natl. Acad. Sci. USA, 89, 10915 (1989); Karlin & Altschul, Proc. Natl. Acad. Sci. USA, 90, 5873-5787 (1993)). Software for performing BLAST analyses is publicly available through the National Center for Biotechnology Information. Other examples of alignment software, including GAP, BESTFIT, FASTA, PILEUP, and TFASTA provided by Wisconsin Genetics Software Package (Genetics Computer Group, 575 Science Dr., Madison, Wis.), and CLUSTALW (Thompson et al., Nuc. Acids Res., 22, 4673-4680 (1994); http://www.ebi.ac.uk/Tools/clustalw2/index.html), are known in the art.

Variation within HCV NS5B sequences can occur as a result of natural mutagenesis, and/or a practitioner can modify an HCV NS5B polymerase polypeptide to create a functional variant falling within the scope of the invention using routine laboratory techniques. For example, in one aspect, any of the polypeptides described herein further comprises a tyrosine at amino acid position 422 as defined in SEQ ID NO: 1. Exemplary amino acid substitutions are those which reduce susceptibility to proteolysis and/or confer or modify other physiochemical or functional properties of the polypeptide. According to certain embodiments, additional amino acid substitutions (for example, conservative amino acid substitutions) are made in the naturally-occurring HCV NS5B sequence. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), beta-branched side chains (e.g., threonine, valine, and isoleucine) and aromatic side chains (e.g., tyrosine, phenylalanine, tryptophan, and histidine). It will be appreciated, however, that a practitioner is not limited to creating conservative substitutions so long as the resulting polypeptide has HCV NS5B polymerase activity.

Amino acid additions and deletions also are appropriate in the context of the invention. For example, in one embodiment, the polypeptide is fused to a heterologous peptide (i.e., amino acids not generally recognized to be part of an HCV NS5B protein sequence). A fusion or chimeric peptide can comprise the entire amino acid sequences of two or more peptides or, alternatively, can be constructed to comprise portions (fragments) of two or more peptides (e.g., 10, 20, 50, 75, 100, 400, 500, or more amino acid residues). It may be desirable to fuse the active domains of two or more factors to generate a fusion peptide having a desired biological activity. In some aspects, the heterologous peptide fused to the inventive polypeptide is, for instance, an immunogenic peptide; a marker protein; a peptide tag, such as a peptide that facilitates purification; a signal peptide; and fragments of any of the foregoing. Exemplary peptide tags include, but are note limited to, a His tag, a FLAG tag, a hemaglutinin tag, a glutathione-S-transferase tag, a green fluorescent protein tag, a maltose binding protein tag, and a chitin binding protein tag.

Removal of one or more amino acid residues from polypeptide also is appropriate. For example, removal of the C-terminal 21 amino acids of the NS5B polyprotein does not destroy polymerase activity, and deletion of 55 residues at the C-terminus has been reported to improve polymerase activity (Liu et al., Biochemistry, 45(38), 11312-11323 (2006); Ivanov et al., Protein Expression and Purification, 48, 14-23 (2006); Ranjith-Kumar and Kao, “Biochemical Activities of the HCV NS5B RNA-Dependent RNA Polymerase” in Hepatitis C Viruses, Tan ed., Horizon Bioscience, Norfolk, UK (2006); pp. 293-310; Ferrari et al., J. Virol., 73, 1649-1654 (1999); Yamashita et al., J. Biol. Chem., 273, 15479-15486 (1998)). The invention embraces HCV NS5B polymerase fragments having the one or more variations described herein. In this regard, the polypeptide fragment comprises at least 100, at least 150, at least 200, at least 250, at least 300, at least 350, at least 400, at least 450, at least 500, or at least 550 amino acids and, in some embodiments, has HCV NS5B polymerase activity. Alternatively, the polypeptide comprises the amino acid sequence of SEQ ID NO: 1 or SEQ ID NO: 3 truncated by 5 or more (e.g., 10 or more, 15 or more, or 20 or more) amino acids at a single terminus or at the N- and C-termini. For example, in one aspect, the polypeptide comprises (or consists of) the amino acid sequence of SEQ ID NO: 1 or SEQ ID NO: 3 (or a similar amino acid sequence having one or more of the variations described herein and, optionally, HCV NS5B polymerase activity) truncated by 25 or more (e.g., 30 or more, 35 or more, 40 or more, 45 or more, 50 or more, 55 or more, or 60 or more) amino acids at a single terminus or at both the N- and C-termini of SEQ ID NOs: 1 or 3. The polypeptide also can comprise the amino acid sequence of SEQ ID NO: 1 or SEQ ID NO: 3 truncated by no more than 5, 10, 15, 20, 25, 30, 35, 40, 45, 50, 55, or 60 amino acids at a single terminus or at both the N- and C-termini. The fragment displays HCV NS5B polymerase activity as described herein.

The polypeptide described herein can be generated using any suitable technique for protein production. In one aspect, the polypeptide is synthesized by solid phase synthesis techniques, solution phase synthesis, or a combination of both techniques, as described in, e.g., Hu, Bioprocessing International, 8(4), 22-25 (2010). In another aspect, the invention includes a method of making a polypeptide of the invention, using a polynucleotide of the invention. For example, the invention includes growing a host cell transformed or transfected with a polynucleotide of the invention under conditions in which the cell expresses the encoded polypeptide. Optionally, the method further includes purifying the polypeptide from the cell or growth media of the cell. Alternatively, the polypeptide can be isolated from a biological sample using, e.g., antibodies or fragments thereof that specifically bind the polypeptide, such as the antibodies described herein.

The polypeptide is chemically modified in some manner distinct from amino acid insertion(s), amino acid deletion(s), or amino acid substitution(s) in some embodiments. In this regard, the polypeptide is chemically bonded with polymers, lipids, other organic moieties, and/or inorganic moieties. Such “peptide derivatives” are prepared to, for instance, increase solubility, absorption, circulating half-life, or targeting to particular cells, tissues, or organs. Suitable modifications include, but are not limited to, attachment to one or more water soluble polymer attachments, such as polyethylene glycol, polyoxyethylene glycol, or polypropylene glycol. Still other useful polymers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of any of the foregoing. In some aspects of the invention, the polypeptide is attached to a detectable label, such as a fluorescent dye (e.g., fluorescein, Alexa, or green fluorescent protein), radioisotope (e.g., ³⁵S, ¹²⁵I, ¹³¹I) or enzyme (e.g., horseradish peroxidase, alkaline phosphatase, secreted alkaline phophatase (SEAP), chloramphenicol acetyltransferase (CAT), luciferase, and β-galactosidase).

Changes in the amino acid sequence of the polypeptide preferably do not substantially adversely affect the structural characteristics associated with HCV NS5B polymerase (e.g., disrupt secondary structure characterizing SEQ ID NO: 1) and/or do not substantially diminish NS5B polymerase activity of the polypeptide. In this regard, one skilled in the art can review amino acid alignments or structure-function studies of similar peptides, such as HCV NS5B polymerases from multiple genotypes, subtypes, or strains, to identify residues in the polypeptide that are important for activity or structure. For example, residues that vary between strains without diminishing activity or likely more susceptible to change, whereas conserved residues are more likely to be important for activity. Mutational analysis of NS5B polymerase is described in, e.g., Qin et al., Hepatology, 33(3), 728-737 (2001). One skilled in the art also can analyze three-dimensional structure and the underlying amino acid sequence responsible for three-dimensional structural domains in similar polypeptides. A number of scientific publications have been devoted to the prediction of secondary structure (Moult, Curr. Op. in Biotech., 7(4), 422-427 (1996); Chou et al., Biochemistry, 13(2), 222-245 (1974); Chou et al., Biochemistry, 113(2), 211-222 (1974); Chou et al., Adv. Enzymol. Relat. Areas Mol. Biol., 47, 45-148 (1978); Chou et al., Ann. Rev. Biochem., 47, 251-276 (1979); Chou et al., Biophys. J., 26, 367-384 (1979); Holm et al., Nucl. Acid. Res., 27(1), 244-247 (1999)). In view of structure information, one skilled in the art predicts the alignment of amino acid residues of a peptide with respect to its three-dimensional structure to determine regions suitable for mutation. In this regard, the three-dimensional structure of NS5B polymerase resembles a right hand, with fingers, palm, and thumb domain organization and an encircled active site (see, e.g., Biswal et al., J. Mol. Biol., 361, 33-45 (2006)). The finger, palm, thumb domain organization of NS5B polymerase is consistent with the structure of other polymerases, among which the palm domain spanning amino acid residues 188-225 and 287-370 is conserved. One skilled in the art may choose not to make radical changes to amino acid residues critical for the finger, palm, thumb domain organization or to amino acid residues predicted to form the catalytic pocket of HCV NS5B polymerase, such as, for example, residues 158, 367, 386, 390, and 394 (see, e.g., Ranjith-Kumar 2006, supra, which is hereby incorporated by reference in its entirety). In some embodiments, radical changes to amino acid sequence are avoided in the longer loop and helix located at the distal portion of the thumb domain and/or avoided in known polymerase motifs (described in Qin et al., supra).

In some embodiments, the polypeptide has HCV NS5B polymerase activity, i.e., effects RNA-dependent RNA synthesis. HCV NS5B polymerase activity can be determined using HCB NS5B polymerase reactions, such as those known in the art and/or described herein. Exemplary HCV NS5B polymerase reactions (i.e., assays) are described in, e.g., Ferrari et al., J. Biol. Chem., 283(49), 33893-33907 (2008), and Behrens et al., EMBO J., 15(1), 12-22 (1996), incorporated herein by reference in their entirety and for their teachings relative to polymerase reactions. For example, one suitable polymerase reaction involves exposing a polypeptide believed to have polymerase activity to RNA template in the presence of nucleotides and measuring the amount of RNA synthesized. The amount of RNA synthesized can be estimated via gel electrophoresis. Colorimetric assays for characterizing RNA polymerase activity also have been described (see, e.g., Lee et al., Bull. Korean Chem. Soc., 30(10), 2485-2488 (2009)). Steady-state kinetic parameters, such as Km, for nucleotide triphosphates and template/primer also are suitable for measuring polymerase activity (see, e.g., McKercher et al., Nucl. Acid Res., 32(2), 422-431 (2004), reporting that, for various NS5B constructs, the Km for UTP ranged from 1.8 to 12 μM and the Km for template/primer ranged from 25 to 214 nM).

RNA synthesis also can be characterized by employing labeled nucleotides (e.g., radiolabeled UTP) to produce a radioactive polymerase reaction product, which can be quantified using, e.g., a scintillation counter (see, e.g., Biswal et al., J. Mol. Biol., 361, 33-45 (2006)). In one embodiment, a scintillation proximity assay is performed, wherein a 5′ biotinylated DNA oligonucleotide (oligo dT) primer (e.g., a primer comprising 15 nucleotides) annealed to a homopolymeric poly rA RNA template is captured on the surface of streptavidin-coated bead. HCV NS5B polymerization activity is quantified by measuring the incorporation of radiolabeled [³H]UTP substrate onto the growing primer 3′ end using, e.g., a liquid scintillation counter.

The results of the HCV NS5B polymerase reaction can be compared to the level of RNA synthesis or kinetic parameters achieved by wild-type HCV NS5B polymerase (e.g., a polymerase comprising the amino acid sequence of SEQ ID NO: 1). In one aspect, the polymerase activity of inventive polypeptide is at least 50% of the polymerase activity demonstrated by a wild-type HCV NS5B polymerase (e.g., at least 60%, at least 70%, at least 75%, at least 80%, at least 85%, at least 90%, at least 95%, or 100% of the activity of wild-type HCV NS5B polymerase). Based on information gathered using polymerase reactions, one skilled in the art can readily determine the amino acids where further sequence variations should be avoided either alone or in combination with other mutations. One can screen any sequence variant to determine whether polymerase activity is retained.

The invention also provides an isolated peptide having no more than 50 amino acids (e.g., no more than 25 amino acids, no more than 15 amino acids, or no more than 10 amino acids) and comprising a sequence of 6-50 amino acids that is at least 90% identical to a portion of the amino acid sequence of SEQ ID NO: 3 encompassing at least one of amino acid residues 419, 423, 482, 486, and 494. When present, the amino acid(s) of the polypeptide that correspond(s) to amino acid residues 419, 423, 482, 486, and/or 494 of SEQ ID NO: 3 is identical to the corresponding amino acid(s) of SEQ ID NO: 3. To illustrate, an exemplary peptide consists of 50 amino acids comprising an amino acid sequence 90% identical to a region of SEQ ID NO: 1 encompassing amino acid position 419 as defined in SEQ ID NO: 1, wherein the amino acid at the position corresponding to position 419 of SEQ ID NO: 1 is cysteine, isoleucine, valine, or proline. Such peptides are useful in a variety of contexts, such as eliciting antibodies that specifically bind to an HCV NS5B polymerase that is resistant to a polymerase inhibitor, such as VX-222, as described in detail herein.

Compositions comprising the inventive polypeptide and a carrier also are specifically contemplated herein. The invention further provides an assay mixture comprising an isolated polypeptide as described herein and one or more components for an HCV polymerase reaction, such as RNA template, RNA primer(s), nucleotide triphosphates, and/or buffer.

### Polynucleotides

The invention further provides an isolated polynucleotide (e.g., a DNA molecule (e.g., cDNA or genomic DNA), RNA molecule (e.g., mRNA), or analog of DNA or RNA)) comprising a nucleic acid sequence encoding any of the polypeptides described herein, e.g., the polypeptide comprising (or consisting of) the amino acid sequence set forth in SEQ ID NOs: 3-502, and compositions comprising the polynucleotides and a carrier. For example, the invention provides an isolated polynucleotide comprising a nucleotide sequence encoding a polypeptide having HCV NS5B polymerase activity, the nucleotide sequence comprising at least one codon variation from SEQ ID NO: 2, the at least one codon variation from SEQ ID NO: 2 selected from the group consisting of: a codon encoding cysteine, isoleucine, valine, or proline at codon 419; a codon encoding alanine at codon 423; a codon encoding alanine, threonine, valine, or asparagine at codon 482; a codon encoding valine, isoleucine, threonine, or serine at codon 486; and a codon encoding isoleucine at codon 494, as the amino acid positions are defined in SEQ ID NO: 2. In various embodiments, the invention provides an isolated polynucleotide comprising a nucleotide sequence encoding a polypeptide having Hepatitis C Virus (HCV) NS5B polymerase activity, the nucleotide sequence comprising at least one codon variation from SEQ ID NO: 2, the at least one codon variation from SEQ ID NO: 2 selected from the group consisting of a codon encoding cysteine, isoleucine, valine, or proline at codon 419; a codon encoding alanine, valine, or asparagine at codon 482; a codon encoding valine, isoleucine, threonine, or serine at codon 486; and a codon encoding isoleucine at codon 494, as the codon positions are defined in SEQ ID NO: 2. For example, the invention provides an isolated polynucleotide comprising a nucleotide sequence that encodes at least one of the amino acid sequences selected from the group consisting of SEQ ID NOs: 4-502.

As noted above with respect to polypeptides, the polynucleotide can comprise a single variation at a codon encoding amino acid position 419, 423, 482, 486, or 496, or can comprise variations at two or more of the codons encoding amino acid positions 419, 423, 482, 486, and/or 496. For example, the polynucleotide can comprise a single variation at a codon encoding amino acid position 419, 482, 486, or 496. All combinations of codon variations encoding cysteine, isoleucine, valine, or proline at amino acid position 419; alanine at codon 423; alanine, threonine, valine, or asparagine at codon 482; valine, isoleucine, threonine, or serine at codon 486; and/or isoleucine at codon 494 are contemplated.

For example, the polynucleotide comprising a codon encoding cysteine, isoleucine, valine, or proline at the position in the nucleotide sequence corresponding to codon 419 of SEQ ID NO: 2 also can comprise: a codon encoding alanine at the position in the nucleotide sequence corresponding to codon 423 of SEQ ID NO: 2 and/or a codon encoding alanine, threonine, valine, or asparagine at the position in the nucleotide sequence corresponding to codon 482 of SEQ ID NO: 2 and/or a codon encoding valine, isoleucine, threonine, or serine at the position in the nucleotide sequence corresponding to codon 486 of SEQ ID NO: 2 and/or a codon encoding isoleucine at the position in the nucleotide sequence corresponding to codon 494 of SEQ ID NO: 2. Similarly, a polynucleotide comprising a codon encoding alanine at the position in the nucleotide sequence corresponding to codon 423 of SEQ ID NO: 2 also can comprise: a codon encoding cysteine, isoleucine, valine, or proline at the position in the nucleotide sequence corresponding to codon 419 of SEQ ID NO: 2 and/or a codon encoding alanine, threonine, valine, or asparagine at the position in the nucleotide sequence corresponding to codon 482 of SEQ ID NO: 2 and/or a codon encoding valine, isoleucine, threonine, or serine at the position in the nucleotide sequence corresponding to codon 486 of SEQ ID NO: 2 and/or a codon encoding isoleucine at the position in the nucleotide sequence corresponding to codon 494 of SEQ ID NO: 2. A polynucleotide comprising a codon encoding alanine, threonine, valine, or asparagine at the position in the nucleotide sequence corresponding to codon 482 of SEQ ID NO: 2 also can comprise: a codon encoding cysteine, isoleucine, valine, or proline at the position in the nucleotide sequence corresponding to codon 419 of SEQ ID NO: 2 and/or a codon encoding alanine at the position in the nucleotide sequence corresponding to codon 423 of SEQ ID NO: 2 and/or a codon encoding valine, isoleucine, threonine, or serine at the position in the nucleotide sequence corresponding to codon 486 of SEQ ID NO: 2 and/or a codon encoding isoleucine at the position in the nucleotide sequence corresponding to codon 494 of SEQ ID NO: 2. A polynucleotide comprising a codon encoding valine, isoleucine, threonine, or serine at the position in the nucleotide sequence corresponding to codon 486 of SEQ ID NO: 2 also can comprise: a codon encoding cysteine, isoleucine, valine, or proline at the position in the nucleotide sequence corresponding to codon 419 of SEQ ID NO: 2 and/or a codon encoding alanine at the position in the nucleotide sequence corresponding to codon 423 of SEQ ID NO: 2 and/or a codon encoding alanine, threonine, valine, or asparagine at the position in the nucleotide sequence corresponding to codon 482 of SEQ ID NO: 2 and/or a codon encoding isoleucine at the position corresponding to 494 of SEQ ID NO: 2. A polynucleotide comprising a codon encoding isoleucine at the position in the nucleotide sequence corresponding to codon 494 of SEQ ID NO: 2 also can comprise: a codon encoding cysteine, isoleucine, valine, or proline at the position in the nucleotide sequence corresponding to codon 419 of SEQ ID NO: 2 and/or a codon encoding alanine at the position in the nucleotide sequence corresponding to codon 423 of SEQ ID NO: 2 and/or a codon encoding alanine, threonine, valine, or asparagine at the position in the nucleotide sequence corresponding to codon 482 of SEQ ID NO: 2 and/or a codon encoding valine, isoleucine, threonine, or serine at the position in the nucleotide sequence corresponding to codon 486 of SEQ ID NO: 2.

In one embodiment, the polynucleotide comprising a codon encoding alanine at the position in the nucleotide sequence corresponding to codon 423 of SEQ ID NO: 2; a codon encoding alanine, threonine, valine, or asparagine at the position in the nucleotide sequence corresponding to codon 482 of SEQ ID NO: 2; a codon encoding valine, isoleucine, threonine, or serine at the position in the nucleotide sequence corresponding to codon 486 of SEQ ID NO: 2; and/or a codon encoding isoleucine at the position in the nucleotide sequence corresponding to codon 494 of SEQ ID NO: 2, also comprises a codon encoding methionine or serine at the position in the nucleotide sequence corresponding to codon 419 of SEQ ID NO: 2. Alternatively, the polynucleotide comprises a codon encoding cysteine, isoleucine, valine, or proline at the position in the nucleotide sequence corresponding to codon 419 of SEQ ID NO: 2; a codon encoding alanine, threonine, valine, or asparagine at the position in the nucleotide sequence corresponding to codon 482 of SEQ ID NO: 2; a codon encoding valine, isoleucine, threonine, or serine at the position in the nucleotide sequence corresponding to codon 486 of SEQ ID NO: 2; and/or a codon encoding isoleucine at the position in the nucleotide sequence corresponding to codon 494 of SEQ ID NO: 2, and further comprises a codon encoding isoleucine, threonine, alanine, or valine at the position in the nucleotide sequence corresponding to codon 423 of SEQ ID NO: 2. In another embodiment, the polynucleotide comprises a codon encoding cysteine, isoleucine, valine, or proline at the position in the nucleotide sequence corresponding to codon 419 of SEQ ID NO: 2; a codon encoding alanine at the position in the nucleotide sequence corresponding to codon 423 of SEQ ID NO: 2; a codon encoding valine, isoleucine, threonine, or serine at the position in the nucleotide sequence corresponding to codon 486 of SEQ ID NO: 2; and/or a codon encoding isoleucine at the position in the nucleotide sequence corresponding to codon 494 of SEQ ID NO: 2, and further comprises a codon encoding leucine or threonine at the position in the nucleotide sequence corresponding to codon 482 of SEQ ID NO: 2. The invention also includes a polynucleotide comprising a codon encoding cysteine, isoleucine, valine, or proline at the position in the nucleotide sequence corresponding to codon 419 of SEQ ID NO: 2; a codon encoding alanine, threonine, valine, or asparagine at the position in the nucleotide sequence corresponding to codon 482 of SEQ ID NO: 2; and/or a codon encoding valine, isoleucine, threonine, or serine at the position in the nucleotide sequence corresponding to codon 486 of SEQ ID NO: 2; and further comprises a codon encoding alanine at the position in the nucleotide sequence corresponding to codon 494 of SEQ ID NO: 2, and, optionally, a codon encoding alanine at the codon position in the nucleotide sequence corresponding to codon 423 of SEQ ID NO: 2. The polynucleotides described herein, in one aspect, further comprises a variation at codon position 422 such that codon encodes tyrosine.

DNA according to this invention may be derived from SEQ ID NO: 2. It will be appreciated that DNA sequences depicted with A, G, C, and T (adenine, guanine, cytosine, and thymine) are intended to also represent equivalent RNA sequences depicted as A, G, C, and U (adenine, guanine, cytosine, and uracil). Methods of preparing DNA and/or RNA molecules are well known in the art. In one aspect, a DNA or RNA molecule encoding a polypeptide provided herein is generated using chemical synthesis techniques and/or using polymerase chain reaction (PCR). The polynucleotide can be isolated from an HCV virus and amplified to provide a population of polynucleotides. In specific embodiments, site-directed mutagenesis of the sequence of SEQ ID NO: 2 is particularly contemplated in order to generate one or more of the polynucleotides described herein.

In a related embodiment, the invention provides an expression vector comprising a polynucleotide of the invention to direct expression of the polynucleotide in a suitable host cell. Such vectors are useful, e.g., for amplifying the polynucleotides in host cells to create useful quantities thereof, and for expressing peptides, such as polymerases, using recombinant techniques. In preferred embodiments, the expression vector comprises the inventive polynucleotide operatively linked to an expression control sequence. Autonomously replicating recombinant expression constructs such as plasmid and viral DNA vectors incorporating the inventive polynucleotides are specifically contemplated. As used herein, “expression vector” is not a native Hepatitis C Virus capable of infection and comprising an unmodified HCV genome. The polynucleotide of the invention is heterologous to the vector and/or linked to an expression control sequence that does not control NS5B polymerase expression in wild-type HCV. Optionally, the expression vector comprises a coding sequence for the inventive polypeptide in the absence of coding sequences for other HCV structural and/or nonstructural proteins (i.e., the expression vector does not comprise a polynucleotide encoding HCV proteins other than NS5B). Also optionally, the expression vector comprises all or part of the 3′ non-translated region of the HCV genome in addition to the polynucleotide of the invention. The 3′ non-translated region has a tripartite structure containing a variable region (comprising approximately 40 nucleotides), a poly (U/UC) tract, and the X tail (comprising approximately 98 nucleotides in length) (Friebe and Bartenschlager, J. Virol., 76(11), 5326-5338 (2002)).

The expression vector can be a viral vector or a non-viral vector (e.g., a plasmid). Exemplary viral vectors include, but are not limited to, retroviral vectors, including lentivirus vectors; parvoviral vectors, such as adeno-associated viral (AAV) vectors; adenoviral vectors; adenoviral adeno-associated chimeric vectors; vaccinia viral vectors; and herpesviral vectors. Any of these expression vectors can be prepared using standard recombinant DNA techniques described in, e.g., Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, a viral vector is rendered replication-deficient by, e.g., deleting or disrupting select genes required for viral replication.

Expression control sequences include promoters, enhancers, and operators, and are generally selected based on the expression systems in which the expression construct is to be utilized. Preferred promoter and enhancer sequences are generally selected for the ability to increase gene expression, while operator sequences are generally selected for the ability to regulate gene expression. Expression constructs may also include sequences encoding one or more selectable markers that permit identification of host cells bearing the construct. Preferred expression constructs also include sequences necessary for replication in a host cell.

Exemplary expression control sequences include promoter/enhancer sequences, e.g., cytomegalovirus promoter/enhancer (Lehner et al., J. Clin. Microbiol., 29, 2494-2502 (1991); Boshart et al., Cell, 41, 521-530 (1985)); Rous sarcoma virus promoter (Davis et al., Hum. Gene Ther., 4, 151 (1993)); simian virus 40 promoter; and albumin promoter, the promoter being operatively linked to the polypeptide coding sequence. The inventive polynucleotides may also optionally include a suitable polyadenylation sequence (e.g., the SV40 or human growth hormone gene polyadenylation sequence) operably linked downstream (i.e., 3′) of the polypeptide coding sequence.

If desired, the polynucleotide of the invention also optionally comprises a nucleotide sequence encoding a secretory signal peptide fused in frame with the polypeptide sequence. The secretory signal peptide directs secretion of the polypeptide of the invention by the cells that express the polynucleotide, and is cleaved by the cell from the secreted polypeptide. The polynucleotide may further optionally comprise sequences whose only intended function is to facilitate large scale production of the vector, e.g., in bacteria, such as a bacterial origin of replication and a sequence encoding a selectable marker.

The invention further provides a cell that comprises the polynucleotide or the expression vector, e.g., the cell is transformed or transfected with a polynucleotide encoding the inventive polypeptide or an expression vector comprising the polynucleotide, and the cell expresses the polypeptide encoded by the polynucleotide. In one aspect, the cell is free of HCV infection. The cell may be a prokaryotic cell, such as Escherichia coli, or a eukaryotic host cell, such as an animal cell (e.g., a mammalian cell, such as a liver cell (hepatocyte), Chinese Hamster Ovary cell, or hybridoma cell), yeast (e.g., Saccharomyces cerevisiae), or a plant cell (e.g., a tobacco, corn, soybean, or rice cell). The host cell may be isolated and/or purified. The host cell may be a primary isolate or a cell from a cell line propagated ex vivo. The host cell may be an isolated cell transformed ex vivo and introduced into an animal post-transformation, e.g., to produce the polypeptide in vivo. The host cell also may be a cell transformed in vivo to cause expression of the polypeptide in vivo. Animal models expressing the inventive polypeptide can include any mammal other than a human, such as rabbits, rodents (e.g., mice, rats, hamsters, gerbils, and guinea pigs), cows, sheep, pigs, goats, horses, dogs, cats, birds (e.g., chickens, turkeys, ducks, and geese), and primates (e.g., chimpanzees, monkeys, and tamarinds).

Certain embodiments of the invention may employ nucleic acid fragments (oligonucleotides) that bind an HCV NS5B coding sequence or a complement thereof. For example, the invention provides an isolated polynucleotide comprising no more than 50 nucleotides and comprising a nucleotide sequence of 14-50 nucleotides complementary to a continuous portion of the nucleotide sequence of any of the polynucleotides described herein, the portion including at least one codon at a codon position selected from codon positions 419, 423, 482, 486, and 494 of SEQ ID NO: 2. In other words, the polynucleotide comprising no more than 50 nucleotides comprises at least one codon variation from SEQ ID NO: 2 selected from the group consisting of: a codon encoding cysteine, isoleucine, valine, or proline at codon 419; a codon encoding alanine at codon 423; a codon encoding alanine, threonine, valine, or asparagine at codon 482; a codon encoding valine, isoleucine, threonine, or serine at codon 486; and a codon encoding isoleucine at codon 494, as the amino acid positions are defined in SEQ ID NO: 2.

Oligonucleotides are useful as primers for the amplification of NS5B nucleic acid molecules. In this regard, oligonucleotides for use in the invention ideally comprise a sufficient number of nucleotide bases to be used in a polymerase chain reaction (PCR) reaction, and can be based on, or designed from, a genomic or cDNA sequence. Oligonucleotides also are useful as hybridization probes to identify (i.e., confirm or reveal) the presence of nucleic acids encoding the inventive polypeptide in a sample. “Probes” refer to nucleic acids derived from any contiguous portion of a nucleic acid sequence of choice. The length of a probe is preferably sufficient for specific hybridization to a nucleic acid sequence encoding NS5B polymerase (e.g., wild-type NS5B or any of the VX-222-resistant NS5B polymerases described herein). In this regard, the polynucleotide fragment is preferably capable of sequence-specific hybridization to a polynucleotide comprising a nucleotide sequence encoding a VX-222-resistant HCV NS5B polymerase, such as a polynucleotide comprising a nucleic acid sequence comprising the codon variations described herein. A nucleic acid probe can comprise as few as 5, 6, 7, 8, 9, or 10 nucleotides that bind to a nucleic acid sequence encoding the inventive polypeptide (or the complement thereof). Preferably, probes are about 15-100 nucleotides in length (e.g., about 15, 20, 25, 30, or 50 nucleotides), although it will be appreciated that probes can comprise as many as about 75, 100, 200, 250, or 500, depending on the desired specificity and conditions of the hybridization reaction.

Short nucleic acid molecules are easily generated synthetically, while longer polynucleotides may be obtained from a natural or recombinant source. Probes may be single- or double-stranded, and preferably are designed to have specificity in PCR, membrane-based hybridization technologies, or ELISA-like technologies. In some embodiments, a probe comprises a detectable label attached thereto, e.g., the probe is labeled with a radioisotope, a fluorescent compound, a biotin-avidin label, an enzyme, or an enzyme co-factor. A non-limiting example of a probe for detecting, e.g., RNA, is a labeled nucleic acid probe of sufficient length to specifically hybridize under stringent conditions to RNA.

### Antibodies

The invention provides an isolated antibody that selectively binds the polypeptide described herein. The term “antibody” refers to a complete (intact) antibody (immunoglobulin) molecule (including polyclonal, monoclonal, chimeric, humanized, or human versions having full length heavy and/or light chains) or an HCV NS5B polymerase-binding fragment thereof. Antibody fragments include F(ab′)2, Fab, Fab′, Fv, Fc, and Fd fragments, and can be incorporated into single domain antibodies, single-chain antibodies, maxibodies, minibodies, intrabodies, diabodies, triabodies, tetrabodies, v-NAR and bis-scFv (see, e.g., Hollinger and Hudson, Nature Biotechnology, 23(9), 1126-1136 (2005)). Antibody polypeptides, including monobodies, also are disclosed in U.S. Pat. No. 6,703,199. Other antibody polypeptides are disclosed in U.S. Patent Publication No. 20050238646.

The term “specifically binds” refers to the ability of the antibody or fragment thereof to bind to an HCV NS5B polymerase having a mutation at one or more of amino acid positions 419, 423, 482, 486, and/or 494 with greater affinity (e.g., at least 10, 15, 20, 25, 50, 100, 250, 500, 1000, or 10,000 times greater affinity) than it binds to an HCV NS5B polymerase having the amino acid sequence of SEQ ID NO: 1. In certain aspects, the antibody binds to an NS5B polymerase polypeptide comprising one or more mutations at amino acid positions 419, 422, 423, 482, 486, and/or 494 with an affinity of less than or equal to 1×10⁻⁷ M, less than or equal to 1×10⁻⁸ M, less than or equal to 1×10⁻⁹ M, less than or equal to 1×10⁻¹⁰ M, less than or equal to 1×10⁻¹¹ M, or less than or equal to 1×10⁻¹² M. Affinity may be determined by an affinity ELISA assay, a BIAcore™ assay (i.e., a surface plasmon resonance-based assay), a kinetic method, or an equilibrium/solution method. Preferably, the antibody distinguishes the HCV NS5B polymerase having one or more variations at positions 419, 423, 482, 486, and/or 494 from wild-type HCV NS5B polymerase, HCV NS5B polymerase not having the one or more variations, or HCV NS5B polymerase that is not resistant to VX-222. For example, in one aspect, the antibody or fragment thereof binds to a polypeptide of the invention with at least 10, 15, 20, 25, 50, 100, 250, 500, 1000, or 10,000 times greater affinity than it binds to an HCV NS5B polymerase not having the one or more amino acid variations described herein, e.g., an NS5B polymerase having the amino acid sequence of SEQ ID NO: 1.

Various procedures are known within the art for producing antibodies, any of which are suitable for production an antibody against an HCV NS5B polymerase having one or more mutations at amino acid positions 419, 423, 482, 486, and/or 494. The antibody or antibody fragment can be isolated from an immunized animal, synthetically made, or genetically-engineered. Antibodies to the inventive polypeptide can be obtained, for example, by immunizing an animal with the inventive polypeptide or fragment thereof, or by introducing into an animal an expression vector encoding the inventive polypeptide or fragment thereof to achieve protein production in vivo. Prior to administration in some instances, a peptide immunogen is covalent coupled to another immunogenic protein, for example, a carrier protein such as keyhole limpet hemocyanin (KLH) or bovine serum albumin (BSA), and/or combined with an adjuvant, such as Freund's complete or incomplete adjuvant. Polyclonal antibodies are typically raised in non-human animals such as rats, mice, rabbits, goats, cattle, or sheep, and also can be raised in a subhuman primate as described in, e.g., International Patent Publication WO 1991/11465 and Losman et al., Int. J. Cancer, 46, 310 (1990). Antibodies raised against a polypeptide of the invention can be screened against an HCV HS5B polymerase having the amino acid sequence of SEQ ID NO: 1 to select those antibodies that bind the polypeptide of the invention with greater affinity then they bind to the polypeptide of SEQ ID NO: 1. In preferred variations, no appreciable cross-reactivity with SEQ ID NO: 1 occurs.

An antibody or fragment thereof also can be genetically-engineered such that the antibody or antibody fragment comprises, e.g., a variable region domain generated by recombinant DNA engineering techniques. For example, a specific antibody variable region can be modified by insertions, deletions, or changes in the amino acid sequence of the antibody to produce an antibody of interest. In this regard, polynucleotides encoding complementarity determining regions (CDRs) of interest are prepared, for example, by using polymerase chain reaction to synthesize variable regions using mRNA of antibody-producing cells as a template (see, for example, Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley-Liss, Inc. 1995); and Larrick et al., Methods: A Companion to Methods in Enzymology, 2, 106-110 (1991)). Antibody manipulation techniques allow construction of engineered variable region domains containing at least one CDR and, optionally, one or more framework amino acids from a first antibody and the remainder of the variable region domain from a second antibody. Such techniques are used, e.g., to humanize an antibody or to improve its affinity for a binding target.

Monoclonal antibodies are generated using a variety of techniques, such as those known in the art (see, for example, Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). In one embodiment, the invention provides an isolated cell capable of producing a monoclonal antibody that selectively binds the polypeptide having NS5B polymerase activity described herein. Typically, monoclonal antibodies are produced by a hybridoma, and the invention provides a hybridoma that produces the inventive monoclonal antibody or antibody fragment. Production of antibodies via immunization of non-human mammals and production of monoclonal antibodies is further described in, e.g., U.S. Pat. No. 7,381,409.

Antibody fragments derived from an intact antibody can be obtained, e.g., by proteolytic hydrolysis of the antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244 (1960); Porter, Biochem. J., 73: 119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1-2.8.10 and 2.10A.1-2.10A.5. Alternatively, such fragments may also be generated by recombinant genetic engineering techniques, such as those techniques known in the art and described herein.

### Screening Methods

The invention further provides a method for detecting the presence of drug-resistant HCV in a sample (e.g., HCV resistant to a polymerase inhibitor, such as VX-222). In one aspect, the method comprises determining the presence or absence of an HCV NS5B polypeptide with an amino acid sequence in which (i) at least one amino acid that corresponds to positions 419, 423, 482, 486, and/or 494 of SEQ ID NO: 3 is identical to the corresponding amino acid(s) of SEQ ID NO: 3, or (ii) at least one amino acid that corresponds to positions 419, 482, 486 and/or 494 of SEQ ID NO: 516 is identical to the corresponding amino acid(s) of SEQ ID NO: 516, wherein the presence of the amino acid(s) in the HCV NS5B protein indicates the presence of drug-resistant HCV. Alternatively or in addition, the method comprises determining the presence or absence of a polynucleotide in the sample, the polynucleotide comprising a nucleic acid sequence encoding HCV NS5B polymerase containing at least one codon selected from the group consisting of a codon encoding cysteine, isoleucine, valine, or proline at a position corresponding to codon position 419 of SEQ ID NO; 2; a codon encoding alanine at a position corresponding to codon 423 of SEQ ID NO: 2; a codon encoding alanine, threonine, valine, or asparagine at a position corresponding to codon 482 of SEQ ID NO: 2; a codon encoding valine, isoleucine, threonine, or serine at a position corresponding to codon 486 of SEQ ID NO: 2; and a codon encoding isoleucine at a position corresponding to codon 494 of SEQ ID NO: 2. For example, in one aspect, the method comprises determining the presence or absence of a polynucleotide comprising a nucleic acid sequence encoding HCV NS5B polymerase containing at least one codon selected from the group consisting of a codon encoding cysteine, isoleucine, valine, or proline at a position corresponding to codon position 419 of SEQ ID NO; 2; a codon encoding alanine, valine, or asparagine at a position corresponding to codon 482 of SEQ ID NO: 2; a codon encoding valine, isoleucine, threonine, or serine at a position corresponding to codon 486 of SEQ ID NO: 2; and a codon encoding isoleucine at a position corresponding to codon 494 of SEQ ID NO: 2. The presence of the codon(s) indicates the presence of drug-resistant HCV in the sample. The sample can be a biological sample, a sample taken from laboratory equipment, and the like.

The invention also provides method for determining whether an HCV-infected patient is infected with an HCV strain that has a decreased sensitivity to VX-222. The method comprises determining the presence or absence of the inventive polypeptide in a biological sample from the patient, wherein the presence of the polypeptide indicates infection with an HCV strain that has a decreased sensitivity to VX-222. Alternatively or in addition, the method comprises determining the presence or absence of the polynucleotide described herein in a biological sample from the patient, wherein the presence of the polynucleotide indicates infection with an HCV strain having a decreased sensitivity to VX-222. In some aspects, one or more samples are taken from a patient over the course of a treatment regimen to detect the emergence of HCV having resistance to a particular polymerase inhibitor, such as VX-222.

In various embodiments, the method for determining whether an HCV-infected patient is infected with an HCV strain that has a decreased sensitivity to VX-222 comprises determining the presence or absence in a sample of a polypeptide comprising an amino acid sequence comprising at least one variation from SEQ ID NO: 1, the at least one variation selected from the group consisting of cysteine, isoleucine, methionine, serine, valine, or proline at amino acid position 419; lysine at amino acid position 422; alanine, isoleucine, threonine, or valine at amino acid position 423; alanine, leucine, threonine, valine, or asparagine at amino acid position 482; valine, isoleucine, threonine, or serine at amino acid position 486; and isoleucine or alanine at amino acid position 494, as the amino acid positions are defined in SEQ ID NO: 1, wherein the presence of the polypeptide indicates infection with an HCV strain that has a decreased sensitivity to VX-222. Alternatively or in addition, the method comprises determining the presence or absence of a polynucleotide in a biological sample patient, the polynucleotide comprising a nucleic acid sequence encoding HCV NS5B polymerase containing at least one codon selected from the group consisting of a codon encoding cysteine, isoleucine, methionine, serine, valine, or proline at a position corresponding to codon position 419 of SEQ ID NO: 2; a codon encoding lysine at a position corresponding to codon position 422 of SEQ ID NO: 2; a codon encoding alanine, isoleucine, threonine, or valine at a position corresponding to codon position 423 of SEQ ID NO: 2; a codon encoding alanine, leucine, threonine, valine, or asparagine at a position corresponding to codon position 482 of SEQ ID NO: 2; a codon encoding valine, isoleucine, threonine, or serine at a position corresponding to codon position 486 of SEQ ID NO: 2; and a codon encoding isoleucine or alanine at a position corresponding to codon position 494 of SEQ ID NO: 2, wherein the presence of the polynucleotide indicates infection with an HCV strain having a decreased sensitivity to VX-222.

In the method, a biological sample obtained from a subject, such as a subject suspected of having or experiencing symptoms associated with HCV infection, a subject undergoing antiviral therapy, or a subject that responds poorly to antiviral therapy. Numerous methods of obtaining biological samples from subject (e.g., a human patient) are available and are appropriate in the context of the invention. Samples typically are isolated from blood, serum, urine, amniotic fluid, or tissue biopsies from, e.g., liver, muscle, connective tissue, or nerve tissue. Once obtained, cells from the sample are examined to detect the presence or absence of the polypeptide or polynucleotide. It will be appreciated that the polypeptide of the invention can be detected in a variety of ways. For example, in one embodiment, NS5B polymerase is isolated from a sample and subjected to amino acid sequencing, the results of which are compared to a reference amino acid sequence, such as the amino acid sequence of wild-type HCV NS5B polymerase (for instance, SEQ ID NO: 1) or HCV NS5B polymerase that is not resistant to VX-222. Immunoassays, e.g., immunofluorescent immunoassays, immunoprecipitations, radioimmunoasays, ELISA, and Western blotting, also can be used to determine the presence or the absence of the polymerase. (See, e.g., Puri et al., J. Virol., 83(13), 6347-6356 (2009).) Thus, in some embodiments, the method comprises contacting the sample with an antibody or fragment thereof that specifically binds the polypeptide and detecting binding of the antibody or fragment thereof to the polypeptide. The antibody or fragment thereof specifically (or preferentially) binds an HCV NS5B polymerase having a mutation at one or more of amino acid positions 419, 423, 482, 486, and 494, such as the antibodies or fragments thereof described herein.

Optionally, the method comprises obtaining nucleic acid sequence data from a biological sample. Indeed, in one aspect, the method further comprises sequencing one or more polynucleotides in the sample to determine the presence the polynucleotide. For many assays, it may be convenient to amplify the NS5B HCV nucleotide (or portion thereof) in the sample using techniques such as PCR. In this regard, a sample of DNA or RNA is obtained, and, if desired, the polynucleotide encoding NS5B polymerase is amplified by PCR. The sample is then examined. Suitable methods of directly analyzing a nucleic acid molecule include, for instance, denaturing high pressure liquid chromatography (DHPLC), DNA hybridization, computational analysis, automated fluorescent sequencing, clamped denaturing gel electrophoresis (CDGE), denaturing gradient gel electrophoresis (DGGE), mobility shift analysis, restriction enzyme analysis, heteroduplex analysis, chemical mismatch cleavage (CMC), RNase protection assays, use of polypeptides that recognize nucleotide mismatches, and direct manual sequencing. These and other methods are described in the art (see, for instance, Tabone et al., Nature Protocols, 1, 2297-2304 (2006); MacBeath et al., DNA Sequencing Protocols, 167, 119-152 (2001) (DOI: 10.1385/1-59259-113-2:119); Li et al., Nucleic Acids Research, 28(2):e1 (i-v) (2000); Liu et al., Biochem. Cell Bio., 80, 17-22 (2000); Burczak et al., Polymorphism Detection and Analysis, Eaton Publishing (2000); Sheffield et al., Proc. Natl. Acad. Sci. USA, 86, 232-236 (1989); Orita et al., Proc. Natl. Acad. Sci. USA, 86, 2766-2770 (1989); Church and Gilbert, Proc. Natl. Acad. Sci. USA, 81, 1991-1995 (1988); Cotton et al., Proc. Natl. Acad. Sci. USA, 85, 4397-4401 (1985); Myers et al., Science, 230, 1242-1246 (1985); Geever et al., Proc. Natl. Acad. Sci. USA, 78, 5081-5085 (1981); Flavell et al., Cell, 15, 25-41 (1978); Sanger et al., Proc. Natl. Acad. Sci. USA, 74, 5463-5467 (1977); and U.S. Pat. No. 5,288,644). In one embodiment, the presence of one or more codon variations is detected directly by sequencing the relevant site(s) of the DNA or RNA in the sample, e.g., regions of the polynucleotide corresponding to codon positions 419, 423, 482, 486, and/or 494 of SEQ ID NO: 2.

In one embodiment, detection of a polynucleotide encoding mutant NS5B polymerase can be accomplished using a hybridization method (see Current Protocols in Molecular Biology, Ausubel et al., eds., John Wiley & Sons (2007), including all supplements). The presence of mutant NS5B can be determined by sequence-specific hybridization of a nucleic acid probe specific for particular mutation within the NS5B polymerase coding sequence. In one aspect, the method comprises contacting polynucleotides in the sample with a nucleic acid probe specific for one or more of the codon variations within the nucleotide sequence of SEQ ID NO: 2 under conditions allowing sequence-specific hybridization of the nucleic acid probe with a target sequence, wherein hybridization of the nucleic acid probe to polynucleotides in the sample indicates the presence of drug-resistance HCV. As discussed above, a nucleic acid probe is a DNA molecule or an RNA molecule that hybridizes to a complementary sequence in genomic DNA, RNA, or cDNA. In some aspects, the presence of more than one codon variation in SEQ ID NO: 2 is determined by using multiple nucleic acid probes, each being specific for a particular variation. In this regard, another variation of the invention is a kit containing 2, 3, 4, 5, 6, or more different polynucleotides of this type, for detecting mutants at two or more of the codons described herein. The inventive method can comprise detecting a non-wild type codon in a position corresponding to codon positions 419, 423, 482, 486, and/or 494 of SEQ ID NO: 2, or combinations thereof, wherein (a) the codon corresponding to codon position 419 of SEQ ID NO: 2 encodes cysteine, isoleucine, valine, or proline; (b) the codon corresponding to codon position 423 of SEQ ID NO: 2 encodes alanine; (c) the codon corresponding to codon position 482 of SEQ ID NO: 2 encodes alanine, threonine, valine, or asparagine; (d) the codon corresponding to codon position 486 of SEQ ID NO: 2 encodes valine, isoleucine, threonine, or serine; (e) and the codon corresponding to codon position 494 of SEQ ID NO: 2 encodes isoleucine.

One of skill in the art has the requisite knowledge and skill to design a probe so that sequence-specific hybridization will occur only if a particular variation is present in a HCV NS5B polymerase coding sequence. By “sequence-specific hybridization” is meant that the probe(s) preferentially bind to a nucleic acid sequence encoding a polypeptide having HCV NS5B polymerase activity and comprising one or more of the variations from SEQ ID NO: 2 described herein. In some embodiments, specific hybridization is achieved using “stringent conditions,” which are conditions for hybridization and washing under which nucleotide sequences at least 60% identical to each other typically remain hybridized. It is appreciated in the art that stringent conditions can differ depending on sequence content, probe length, and the like. Generally, stringent conditions are selected to be about 5° C. lower than the thermal melting point (Tm) for a specific sequence at a defined ionic strength and pH. Tm is the temperature (under defined ionic strength, pH, and nucleic acid concentration) at which 50% of the probes complementary to the target sequence hybridize to the target sequence at equilibrium. Since target sequences are generally present at excess, 50% of the probes are occupied at equilibrium at Tm. Stringent conditions also may include a salt concentration less than about 1.0 M sodium ion, typically about 0.01 to 1.0 M sodium ion (or other salts) at pH 7.0 to 8.3 and the temperature is at least about 30° C. for short probes, primers, or oligonucleotides (e.g., 10 nucleotides to 50 nucleotides) and at least about 60° C. for longer probes, primers and oligonucleotides. Stringent conditions may also be achieved with the addition of destabilizing agents, such as formamide. A non-limiting example of stringent hybridization conditions are hybridization in a high salt buffer comprising 6×SSC, 50 mM Tr-is-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C.

Specific hybridization, if present, is detected using standard methods. For example, the probe can comprise a fluorescent moiety at its 3′ terminus, a quencher at its 5′ terminus, and an enhancer oligonucleotide to facilitate detection, as described by Kutyavin et al., Nucleic Acid Res., 34:e128 (2006). In this detection method, an enzyme cleaves the fluorescent moiety from a fully complementary detection probe, but does not cleave the fluorescent moiety if the probe contains a mismatch. The presence of a particular target sequence is signaled by the fluorescence of the released fluorescent moiety. Alternatively, polynucleotides from a sample are dot-blotted using standard methods, and the blot is contacted with one or more oligonucleotide probes specific for a variation conferring drug-resistance (see, for example, Saiki et al., Nature, 324, 163-166 (1986)). Similarly, arrays of oligonucleotide probes complementary to target nucleic acid sequence(s) can be employed. Oligonucleotide arrays typically comprise a plurality of different oligonucleotide probes coupled to a surface of a substrate (e.g., plastic, complex carbohydrate, or acrylic resin) in different known locations. Such arrays are generally produced using mechanical synthesis methods or light-directed synthesis methods, although other methods are known to the ordinary skilled practitioner (see, e.g., Lau et al., Hong Kong Med. J., 14(5), Suppl. 5, 4-7 (2008); Bier et al., Adv. Biochem. Eng. Biotechnol., 109, 433-53 (2008); Hoheisel, Nat. Rev. Genet., 7, 200-10 (2006); Fan et al., Methods Enzymol., 410, 57-73 (2006); Raqoussis & Elvidge, Expert Rev. Mol. Diagn., 6, 145-52 (2006); Mockler et al., Genomics, 85, 1-15 (2005), and references cited therein, the entire teachings of each of which are incorporated by reference herein). In another hybridization method, Northern analysis (see Current Protocols in Molecular Biology, Ausubel et al., eds., John Wiley & Sons (2007)) is used to detect RNA encoding mutant HCV NS5B polymerase in a sample. Specific hybridization between the nucleic acid probe and the polynucleotide in the sample indicates that drug-resistant HCV (e.g., HCV having resistance to a polymerase inhibitor, such as VX-222) is present.

An exemplary method of detecting HCV having resistance to a polymerase inhibitor is the cycling probe method described in, e.g., Suzuki et al., J. Clin. Microbiol., 48(1), 57-63 (2010). Cycling probe technology can detect single nucleotide polymorphisms in a target DNA sequence by using probe-adapted real-time PCR. The probe accommodates an RNA complementary to the target DNA that undergoes degeneration by RNase H once a DNA-RNA complex is formed. Two cycling probes labeled with different fluorescent dyes and a quencher are used to detect SNPs, each probe harboring RNA corresponding to the wild-type polynucleotide or the polynucleotide having the mutation of interest. When a probe fully anneals to a target sequence, RNase cleaves the probe, releasing the fluorescent dye from the quencher for detection. Cleavage by RNase does not occur where there is a mismatch between the probe and the target sequence, thereby allowing detection of variations at certain positions within a nucleotide sequence. (Suzuki et al., supra.)

Other assays appropriate for detecting mutant HCV in a sample include, but are not limited to, the restriction fragment length polymorphism (RFLP) assay, the 5′-nuclease (TaqMan) assay, the TaqMAMA assay, and ligase detection reaction (LDR), which are capable of detecting a single-nucleotide change in polymerase proteins, as described in, e.g., Shafer et al., J. Clin. Microbiol., 34(7), 1849-1853 (1996) (LDR); Allen et al., J. Clin. Microbiol., 37, 3338-3347 (1999) (RFLP and TaqMan); Li et al., Genomics, 83, 311-320 (2004) (TaqMAMA) (all of which are herein incorporated by reference in their entirety and specifically with respect to the descriptions of methods of detecting mutant polynucleotides). The RFLP assay is a PCR-based assay employing restriction endonucleases that cleave specific sequences within a target nucleic acid sequence, resulting in a specific set of DNA fragments. Changes in the DNA pattern visualized using, e.g., polyacrylamide gel electrophoresis, indicates one or more mutations within the nucleic acid sequence (Allen et al., supra). Similarly, the 5′-nuclease assay is a PCR-based assay that utilizes the exonuclease activity of Taq DNA polymerase to cleave a probe labeled with a fluorescent dye and quencher. When the probe hybridizes to a target sequence, Taq cleaves the probe to release the dye from the quencher, thereby generating a detectable signal (Allen et al., supra). TaqMAMA is an allele-specific PCR-based assay wherein PCR products are preferentially formed from DNA that contains a desired target nucleic acid sequence (Li et al., supra).

The invention also provides methods of using the polymerase (or polynucleotide encoding the polymerase) to, e.g., identify polymerase inhibitors. The term “polymerase inhibitor” as used herein means an agent (compound or biological) that is effective to inhibit the function of HCV polymerase in a mammal, such as a human or a non-human mammal. The invention encompasses a method for characterizing the HCV inhibitory activity of an agent. The method comprises performing an HCV NS5B polymerase reaction with the inventive polypeptide in the presence of an agent, and comparing polymerase activity in the presence of the agent with polymerase activity of the polypeptide in the absence of the agent. Optionally, the method further comprises performing an HCV NS5B polymerase reaction with the inventive polypeptide in the absence of the agent. HCV HS5B polymerase reactions are described above, and the effect of an agent on any parameter indicative of polymerase activity is appropriate. For example, in one embodiment, comparing polymerase activity comprises comparing the amount of polynucleotide generated by the NS5B polymerase reaction with the polypeptide in the presence of the agent with the amount of polynucleotide generated by the reaction in the absence of the agent, wherein a decrease in the amount of polynucleotide generated by the polypeptide in the presence of the agent is indicative of HCV inhibitory activity of the agent.

An exemplary assay for detecting the inhibitory effect of an agent on HCV NS5B polymerase uses the MultiScreen™ assay format, which evaluates the amount of radiolabeled UTP incorporated by a polymerase into a newly synthesized RNA using a homopolymeric RNA template/primer. Generally, agents are tested at a variety of concentrations in a reaction mixture comprising buffer (e.g., 20 mM Tris-HCl, pH 7.5, 5 mM MgCl₂, 1 mM DTT, 50 mM NaCl), polymerase (e.g., 400 ng of purified NS5B polymerase enzyme), and labeled nucleotides (e.g., 500 ng of polyrA/oligodT₁₅) (Canadian Life Technologies, Burlington, Ontario, Canada). RNA-dependent-RNA polymerase reactions are allowed to proceed for, e.g., 140 minutes at 22° C., after which the reactions are stopped by the addition of, e.g., 10 μL of 0.5 mM EDTA. Thereafter, a volume of 50 μL (25 μg) of sonicated salmon sperm DNA and 100 μL of a solution of 20% trichloroacetic acid-0.5% tetrasodium pyrophosphate at 4° C. is added to the mixture, which is incubated on ice for 30 minutes to ensure complete precipitation of nucleic acids. Samples are then transferred onto 96-well MultiScreen™ filter plates (Millipore Corp., Bedford, Mass., USA). The filter plates are washed with 600 μL 1% trichloroacetic acid-0.1% tetrasodium pyrophosphate per well, and dried 20 minutes at 37° C. A 50 μL volume of liquid scintillation cocktail (Wallac Oy, Turku, Finland) is added and the incorporated radioactivity is quantified using a liquid scintillation counter (Wallac MicroBeta Trilux, Perkin Elmer™, MA, USA). The reaction conditions described herein are exemplary and can be modified as needed by a practitioner.

The polypeptide having NS5B polymerase activity and one or more variations selected from the group consisting of cysteine, isoleucine, valine, methionine, serine, or proline at amino acid position 419; lysine at amino acid position 422; alanine, isoleucine, threonine, or valine at amino acid position 423; alanine, threonine, leucine, valine, or asparagine at amino acid position 482; valine, isoleucine, threonine, or serine at amino acid position 486; and isoleucine or alanine at amino acid position 494 (as the amino acid positions are defined in SEQ ID NO: 1), displays resistance against at least one polymerase inhibitor, such as VX-222. VX-222 is a NS5B polymerase inhibitor, described further in WO 2008/058393 and WO 2002/100851 (incorporated herein by reference in their entirety and particularly with respect to the description of polymerase inhibitors), represented by Formula (I) (free acid form), and its pharmaceutically acceptable salts, prodrugs, and solvates thereof.

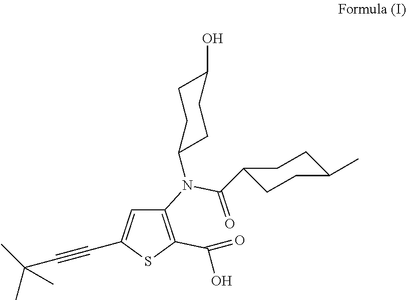 [SMILES: CC1CCC(C(=O)N(c2cc(C#CC(C)(C)C)sc2C(=O)O)C2CCC(O)CC2)CC1]

As used herein, the phrase “pharmaceutically acceptable salt(s)” refers to the salts that are safe and effective for treatment of HCV infections. “Pharmaceutically acceptable salts” include, e.g., those derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts. Salts derived from amino acids are also included (e.g. L-arginine, L-Lysine). Salts derived from appropriate bases include alkali metals (e.g. sodium, lithium, potassium), alkaline earth metals (e.g. calcium, magnesium), ammonium, NR4+ (where R is C1-4 alkyl) salts, aluminum, zinc, diethanolamine salts, choline and tromethamine. Pharmaceutically acceptable salts with various amino acids can also be used, and use of these amino acid salts is also within the scope of this invention. Suitable salts include, but are not limited to, sodium salt, lithium salt, potassium salt, tromethamine salt, hydrochloride salt, hydrobromide salt, hydroiodide salt, nitrate salt, sulfate salt, bisulfate salt, phosphate salt, acid phosphate salt, isonicotinate salt, acetate salt, lactate salt, and L-arginine salt. For a review on pharmaceutically acceptable salts, see Berge et al., J. Pharm. Sci., 66, 1-19 (1977), the contents of which are incorporated herein by reference.

As used herein, the phrase a “pharmaceutically acceptable prodrug” of VX-222 refers to a compound that may be converted under physiological conditions or by solvolysis to VX-222 or to a pharmaceutically acceptable salt of VX-222 prior to exhibiting its pharmacological effect in the treatment of HCV infections.

As used herein, the phrase a “pharmaceutically acceptable solvate” of VX-222 refers to a pharmaceutically acceptable solvate form of VX-222 that contains solvent molecule(s) and retains the biological effectiveness of VX-222. In the case of salts, prodrugs, or solvates of VX-222 that are solids, it is understood by those skilled in the art that these salts, prodrugs, and solvates may exist in different crystalline or amorphous forms, the use of all of which is also within the scope of the present invention.

The polypeptide may also have resistance to compounds that differ from VX-222 only in the presence of one or more isotopically enriched atoms. For example, compounds having the structure of Formula I except for the replacement of hydrogen by deuterium or tritium, or the replacement of a carbon by a 13C- or 14C-enriched carbon are contemplated. VX-222 may independently contain one or more asymmetric carbon atoms and thus may occur as racemates and racemic mixtures, single enantiomers, diastereomeric mixtures and individual diastereomers. All such isomeric forms of these compounds are expressly included in the present invention. Each stereogenic carbon may be of the R or S configuration.

A method for identifying an agent able to rescue or enhance the polymerase-inhibitory activity of VX-222 against an HCV NS5B polymerase having resistance to VX-222 is provided. The method comprises a) performing an HCV NS5B polymerase reaction with the inventive polypeptide in the presence of an agent and VX-222; and b) comparing polymerase activity of the polypeptide in the presence of the agent with polymerase activity of the polypeptide in the absence of the agent, wherein a decrease in HCV polymerase activity in the presence of the agent is indicative of the ability to rescue the polymerase-inhibitory activity of VX-222. In at least one aspect, the invention provides a method for identifying an agent able to rescue or enhance the polymerase-inhibitory activity of VX-222 against a resistant HCV NS5B polymerase comprising performing an HCV NS5B polymerase reaction with a polypeptide comprising an amino acid sequence comprising at least one variation from SEQ ID NO: 1, the at least one variation selected from the group consisting of cysteine, isoleucine, methionine, serine, valine, or proline at amino acid position 419; lysine at amino acid position 422; alanine, isoleucine, threonine, or valine at amino acid position 423; alanine, leucine, threonine, valine, or asparagine at amino acid position 482; valine, isoleucine, threonine, or serine at amino acid position 486; and isoleucine or alanine at amino acid position 494, as the amino acid positions are defined in SEQ ID NO: 1, in the presence of an agent and VX-222; and b) comparing polymerase activity of the polypeptide in the presence of the agent with polymerase activity of the polypeptide in the absence of the agent.

The invention also provides compositions that comprise the agents identified in the methods described herein to rescue or enhance the polymerase inhibitor activity of VX-222 and/or display HCV inhibitory activity, and uses of the agents. Such compositions may be used to pre-treat invasive devices to be inserted into a patient, to treat biological samples, such as blood, prior to administration to a patient, and for direct administration to a patient. The composition can be used to inhibit HCV replication and to lessen the risk of or the severity of HCV infection. The composition comprises the agent and a carrier, such as a pharmaceutically-acceptable carrier. According to a preferred embodiment, the agent is present in the composition in an amount effective to decrease the viral load in a sample or in a patient.

The agents utilized in the compositions and methods described herein may also be modified by appending appropriate functionalities to enhance selective biological properties. Such modifications are known in the art and include those which increase biological penetration into a given biological system (e.g., liver, blood, lymphatic system, and/or central nervous system), increase oral availability, increase solubility to facilitate administration by injection, alter metabolism, and/or alter rate of excretion.

It will be appreciated that the amount of an agent described herein for use in treatment will vary not only with the particular agent selected but also with the route of administration, the nature of the condition for which treatment is required and the age and condition of the subject. In general, however, a suitable dose will be in the range of from about 0.1 to about 750 mg/kg of body weight per day, for example, in the range of 0.5 to 60 mg/kg/day, or, for example, in the range of 1 to 20 mg/kg/day. The agent is conveniently administered in unit dosage form, for example containing 10 to 1500 mg, conveniently 20 to 1000 mg, most conveniently 50 to 700 mg of active ingredient per unit dosage form. The desired dose may be presented in a single dose or as divided doses administered at appropriate intervals, for example as two, three, four, five or more doses per day. The amount of active ingredient that may be combined with the carrier materials to produce a single dosage form will vary depending upon the subject and the particular mode of administration. A typical preparation will contain from about 5% to about 95% active compound (w/w), e.g., from about 20% to about 80% active compound.

In some embodiments, the agent should be administered to achieve peak plasma concentrations of the active ingredient of from about 1 to about 75 μM, about 2 to 50 μM, about 3 to about 30 μM. This may be achieved, for example, by the intravenous injection of a 0.1 to 5% solution of the active ingredient, optionally in saline, or orally administered as a bolus containing about 1 to about 500 mg of the active ingredient. Desirable blood levels may be maintained by a continuous infusion to provide about 0.01 to about 5.0 mg/kg/hour or by intermittent infusions containing about 0.4 to about 15 mg/kg of the active ingredient.

When the agents of the invention or pharmaceutically acceptable salts thereof are used in combination with a second therapeutic agent active against the same virus, the dose of each compound may be either the same as or differ from that when the agent is used alone.

While it is possible that, for use in therapy or treatment of objects, an agent may be administered as the raw chemical it is preferable to present the agent as a pharmaceutical composition. The invention further provides a pharmaceutical composition comprising one or more agents described herein or a pharmaceutically acceptable derivative thereof, together with one or more pharmaceutically acceptable carriers and, optionally, other therapeutic and/or prophylactic ingredients. The carrier(s) must be “acceptable” in the sense of being compatible with the other ingredients of the formulation and not deleterious to the recipient thereof.

Pharmaceutical compositions suitable for oral administration may conveniently be presented as discrete units such as capsules, cachets or tablets each containing a predetermined amount of the active ingredient; as a powder or granules; as a solution, a suspension or as an emulsion. The agent may also be presented as a bolus, electuary or paste. Tablets and capsules for oral administration may contain conventional excipients such as binding agents, fillers, lubricants, disintegrants, or wetting agents. The tablets may be coated. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The agents described herein may also be formulated for parenteral administration (e.g. by injection, for example bolus injection or continuous infusion) and may be presented in unit dose form in ampules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the agent may be in powder form, obtained by aseptic isolation of sterile solid or by lyophilization from solution, for constitution with a suitable vehicle, e.g. sterile, pyrogen-free water, before use. A sterile injectable preparation may be a solution or suspension in a non-toxic parenterally-acceptable diluent or solvent, for example as a solution in 1,3-butanediol. Among the acceptable vehicles and solvents that may be employed are water, Ringer's solution, and isotonic sodium chloride solution. In addition, sterile, fixed oils are employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. Oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans, and other emulsifying agents or bioavailability enhancers which are commonly used in the manufacture of pharmaceutically acceptable solid, liquid, or other dosage forms may also be used for the purposes of formulation.

For topical administration to the epidermis, the agents described herein may be formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Compositions suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the agent in a suitable liquid carrier.

Pharmaceutical compositions suitable for rectal administration wherein the carrier is a solid are for example presented as unit dose suppositories. Suitable carriers include cocoa butter and other materials commonly used in the art, and the suppositories may be conveniently formed by admixture of the agent with the softened or melted carrier(s) followed by chilling and shaping in moulds. Compositions suitable for vaginal administration may be presented as pessaries, tampons, creams, gels, pastes, foams or sprays containing in addition to the agent such carriers as are known in the art to be appropriate.

For intra-nasal administration the agents may be incorporated into a liquid spray or dispersible powder or in the form of drops. Drops may be formulated with an aqueous or non-aqueous base also comprising one more dispersing agents, solubilizing agents or suspending agents. Liquid sprays are conveniently delivered from pressurized packs. For administration by inhalation, the agents are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

Alternatively, for administration by inhalation or insufflation, the agent may take the form of a dry powder composition, for example a powder mix of the compound and a suitable powder base such as lactose or starch. The powder composition may be presented in unit dosage form in, for example, capsules or cartridges or e.g. gelatin or blister packs from which the powder may be administered with the aid of an inhalator or insufflator.

When desired the above described formulations adapted to give sustained release of the active ingredient may be employed.

In one embodiment, the invention provides a pharmaceutical composition comprising at least one agent described herein, which is administered to a subject or exposed to an object in combination with at least one additional agent chosen from viral serine protease inhibitors, viral polymerase inhibitors, viral helicase inhibitors, immunomodulating agents, antioxidant agents, antibacterial agents, therapeutic vaccines, hepatoprotectant agents, antisense agents, inhibitors of HCV NS2/3 protease, and inhibitors of internal ribosome entry site (IRES).

In another embodiment, there is provided a composition comprising a least one agent described herein and one or more additional agents chosen from viral serine protease inhibitors, viral polymerase inhibitors, viral helicase inhibitors, immunomodulating agents, antioxidant agents, antibacterial agents, therapeutic vaccines, hepatoprotectant agents, antisense agent, inhibitors of HCV NS2/3 protease and inhibitors of internal ribosome entry site (IRES).

In one combination embodiment, the compound and additional agent are administered sequentially. In another combination embodiment, the compound and additional agent are administered simultaneously. The combinations referred to herein may conveniently be presented for use in the form of a pharmaceutical formulation. Pharmaceutical formulations comprising a combination of the agent and at least one other active ingredient with a pharmaceutically acceptable carrier comprise a further aspect of the invention. The additional agents for the compositions and combinations include, for example, Ribavirin, amantadine, merimepodib, Levovirin, Viramidine, and maxamine.

The term “viral serine protease inhibitor” as used herein means an agent that is effective to inhibit the function of the viral serine protease including HCV serine protease in a mammal. Indeed, in one embodiment, viral serine protease inhibitor is a flaviviridae serine protease inhibitor. Inhibitors of HCV serine protease include, for example, those compounds described in International Patent Publication Nos. WO 1999/07733 (Boehringer Ingelheim), WO 1999/07734 (Boehringer Ingelheim), WO 2000/09558 (Boehringer Ingelheim), WO 2000/09543 (Boehringer Ingelheim), WO 2000/59929 (Boehringer Ingelheim), WO 2002/060926 (BMS), WO 2006/039488 (Vertex), WO 2005/077969 (Vertex), WO 2005/035525 (Vertex), WO 2005/028502 (Vertex) WO 2005/007681 (Vertex), WO 2004/092162 (Vertex), WO 2004/092161 (Vertex), WO 2003/035060 (Vertex), WO 2003/087092 (Vertex), WO 2002/18369 (Vertex), and WO 1998/17679 (Vertex). Specific examples of inhibitors of HCV NS3 protease include, but are not limited to, BILN-2061 (Boehringer Ingelheim) SCH-6 and SCH-503034/Boceprevir (Schering-Plough), VX-950/telaprevir (Vertex), ITMN-B (InterMune), GS9132 (Gilead), TMC-435350 (Tibotec/Medivir), ITMN-191 (InterMune), and MK-7009 (Merck) (all of which are hereby incorporated by reference in their entirety and particularly with respect to the discussion of protease inhibitors).

The term “viral polymerase inhibitors” as used herein means an agent that is effective to inhibit the function of a viral polymerase, including an HCV polymerase in a mammal. In one embodiment, viral polymerase inhibitor is a flaviviridae polymerase inhibitor. Inhibitors of HCV polymerase include non-nucleosides, for example, those compounds described in International Patent Publication Nos. WO 2003/010140 (Boehringer Ingelheim), WO 2003/026587 (Bristol Myers Squibb); WO 2002/100846, WO 2002/100851, WO 2001/85172 (GSK), WO 2002/098424 (GSK), WO 2000/06529 (Merck), WO 2002/06246 (Merck), WO 2001/47883 (Japan Tobacco), WO 2003/000254 (Japan Tobacco), and EP 1 256 628 A2 (Agouron). Other inhibitors of HCV polymerase include, for example, nucleoside analogs, such as those compounds described in International Patent Publication Nos. WO 2001/90121 (Idenix), WO 2002/069903 (Biocryst Pharmaceuticals Inc.), WO 2002/057287 (Merck/Isis), and WO 2002/057425 (Merck/Isis) (all of which are hereby incorporated by reference in their entirety and particularly with respect to the discussion of polymerase inhibitors). Specific examples of nucleoside inhibitors of an HCV polymerase, include R1626/R1479 (Roche), R7128 (Roche), MK-0608 (Merck), R1656 (Roche-Pharmas set), and Valopicitabine (Idenix). Other specific examples of inhibitors of HCV polymerase include, but are not limited to, JTK-002/003 and JTK-109 (Japan Tobacco), HCV-796 (Viropharma), GS-9190 (Gilead), and PF-868,554 (Pfizer).

The term “viral helicase inhibitors” refers to an agent that is effective to inhibit the function of a viral helicase, including a Flaviviridae helicase in a mammal. Indeed, in one embodiment, viral helicase inhibitor is a flaviviridae helicase inhibitor.

“Immunomodulatory agent” means those agents that are effective to enhance or potentiate the immune system response in a mammal. Immunomodulatory agents include, for example, class I interferons (such as α-, β-, δ- and Ω-interferons, x-interferons, consensus interferons and asialo-interferons), class II interferons (such as γ-interferons) and pegylated interferons. The term “class I interferon” as used herein means an interferon selected from a group of interferons that all bind to receptor type 1. This includes both naturally and synthetically produced class I interferons. Examples of class I interferons include, but are not limited to, α-, β-, δ- and Ω-interferons, τ-interferons, consensus interferons and asialo-interferons. The term “class II interferon” as used herein means an interferon selected from a group of interferons that all bind to receptor type II. Examples of class II interferons include γ-interferons. Interferon is available in pegylated and non pegylated forms. Pegylated interferons include PEGASYS™ and Peg-intron™

Antisense agents also may be administered in conjunction with the agent described herein. Antisense agents include, for example, ISIS-14803. Inhibitors of internal ribosome entry sites (IRES) include ISIS-14803 (ISIS Pharmaceuticals) and compounds described in International Patent Publication No. WO 2006/019831 (PTC therapeutics) (which is hereby incorporated by reference in its entirety and particularly with respect to the discussion of antisense).

In one embodiment, the additional active agent administered with the agent described herein is interferon α, ribavirin, silybum marianum, interleukin-12, amantadine, ribozyme, thymosin, N-acetyl cysteine or cyclosporin. In one embodiment, the additional agent is interferon α 1A, interferon α 1 B, interferon α 2A, or interferon α 2B.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh. The recommended dose of PEGASYS™ when used in combination with ribavirin for chronic hepatitis C is 180 mg (1.0 mL vial or 0.5 mL prefilled syringe) once weekly. The recommended dose of PEG-Intron™ regimen is 1.0 mg/kg/week subcutaneously for one year. The dose should be administered on the same day of the week. When administered in combination with Ribavirin, the recommended dose of PEG-Intron™ is 1.5 micrograms/kg/week. The daily dose of Ribavirin is 800 mg to 1200 mg administered orally in two divided doses. The dose should be individualized to the patient depending on baseline disease characteristics (e.g., genotype), response to therapy, and tolerability of the regimen.

The drug combinations of the present invention can be provided to a cell or cells, or to a human patient, either in separate pharmaceutically acceptable formulations administered simultaneously or sequentially, formulations containing more than one therapeutic agent, or by an assortment of single agent and multiple agent formulations. Regardless of the route of administration, these drug combinations form an anti-HCV effective amount of components of the pharmaceutically acceptable formulations.

Upon improvement of a patient's condition, a maintenance dose of an agent, composition or therapeutic combination may be administered, if desired. Subsequently, the dosage or frequency of administration, or both, may be reduced, as a function of the symptoms, to a level at which the improved condition is retained when the symptoms have been alleviated to the desired level, treatment should cease. Patients may, however, require intermittent treatment on a long-term basis upon any recurrence of disease symptoms.

According to another embodiment, the invention provides a method for treating a patient infected with a virus characterized by a virally encoded polymerase that is necessary for the life cycle of the virus by administering to said patient a pharmaceutically acceptable composition of this invention. Preferably, the virus is HCV and/or the patient is a human. Such treatment may completely eradicate the viral infection or reduce the severity thereof.

In yet another embodiment, a method of pre-treating a biological substance intended for administration to a patient is provided. The method comprises contacting the biological substance with a pharmaceutically acceptable composition comprising an agent identified as described herein. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

A method of treating materials that may potentially come into contact with a virus characterized by a virally encoded polymerase necessary for its life cycle also is contemplated. The method comprises contacting the material with an agent identified as described herein. Such materials include, but are not limited to, surgical instruments and garments; laboratory instruments and garments; blood collection apparatuses and materials; and invasive devices, such as shunts and stents.

In another embodiment, the agent of the invention is a laboratory tool for isolating viral polymerase, such as HCV NS5B. The agent is attached to a solid support, which is contacted with a sample containing a viral polymerase under conditions that cause said polymerase to bind to the solid support. The polymerase is then eluted from the solid support. In one embodiment, the polymerase is a mutant HCV NS5B polymerase that is resistant to treatment by VX-222 as described herein. Exemplary polymerases includes those described herein as having variations at positions 419, 423, 482, 486, and/or 494 of SEQ ID NO: 1.

The invention is further described in the following examples. The examples serve only to illustrate the invention and are not intended to limit the scope of the invention in any way.

### EXAMPLES

### Example 1

This example describes a method of detecting polynucleotides encoding HCV NS5B polymerase and having one or more of the codon variations described herein.

VX-222 is a non-nucleoside hepatitis C virus (HCV) NS5B polymerase inhibitor with potent in vitro activity. Safety, antiviral activity, and viral sequences were assessed in a phase Ib/IIa multicenter, randomized, dose-ascending study in genotype 1, HCV-infected patients.

Treatment-naïve HCV genotype 1 patients (n=32) were randomized to receive VX-222 at doses of 250 mg BID, 500 mg BID, 750 mg BID, or 1500 mg QD for 3 days, in a treatment:placebo ratio of 6:2 (8 patients/cohort). Pegylated interferon (Peg-IFN) and ribavirin (RBV) for 48 weeks was offered to patients at the end of the study. Sequencing of NS5B was performed at baseline (Day 1), end of dosing (Day 3), and at follow-up timepoints (Days 4, 5, 10, 20, and 60). Phenotypic data (fold change in IC50 from wildtype) of substitutions selected in patients during dosing was determined using a replicon system.

The mean HCV RNA decline achieved after three days of dosing with 250 mg, 500 mg, 750 mg, and 1500 mg of VX-222 was 3.1, 3.4, 3.2, and 3.4 log 10, respectively. The most common reported adverse effects (AEs) were diarrhea, headache, nausea, fatigue and fever. The majority of AEs were mild in severity.

NS5B polynucleotide was directly sequenced from all subjects at all time points. No subtype dependent differences were identified in the variants. In other words, the genotype 1a and genotype 1b viruses contained the same variations in amino acid sequence. Sequencing results showed substitutions at 6 positions in NS5B polymerase at the end of dosing: substitution of cysteine, methionine, proline, serine, or valine for leucine at amino acid position 419 (L419C/M/P/S/V); substitution of lysine for arginine at amino acid position 422 (R422K); substitution of isoleucine, threonine, or valine for methionine at amino acid position 423 (M423I/T/V); substitution of leucine, asparagine, or threonine for isoleucine at amino acid position 482 (1482L/N/T); substitution of isoleucine or valine for alanine at amino acid position 486 (A486I/V); and substitution of alanine for valine at amino acid position 494 (V494A). Phenotypic analyses of these variants showed a range of decreased sensitivity to VX-222 (˜6-80 fold change in IC50). For example, substitution at position 494 resulted in about a six-fold change in IC50, substitution at position 423 resulted in about a seven to ten-fold change in IC50, substitution at position 482 resulted in about a 26-fold change in IC50, substitution at position 419 resulted in about a 20-80-fold change in IC50, substitution at position 486 resulted in about a 50-fold change in IC50, and substitution at position 422 resulted in about a 74-fold change in IC50. The majority of subjects who were above the limit of detection of the sequencing assay had variants at the end of dosing. In most patients, only wild-type virus was detected at Day 10 of follow-up. Variants did not have reduced sensitivity to IFN, RBV, or other classes of STAT-Cs tested (e.g., polymerase or protease inhibitors) in vitro.

VX-222 produced a mean HCV RNA decline of greater than 3 log 10 in all four dose groups. The most common reported AEs were diarrhea, headache, nausea, fatigue and fever. Substitutions associated with decreased sensitivity to VX-222 were observed in the NS5B gene in the majority of patients after dosing with VX-222. While the fold change in IC50 to VX-222 conferred by these substitutions varied, they remained sensitive to IFN, RBV, and other classes of STAT-Cs. Variants were less fit than wild-type virus, and in the majority of patients the viral population returned to wild-type virus during the follow-up period.

### Example 2

This example describes the construction of an exemplary expression vector for producing HCV NS5B polymerase protein.

An HCV NS5B polymerase (genotype 1a) having a C-terminal truncation of 21 amino acids was amplified by PCR using the Vent DNA polymerase (New England BioLabs Inc., Mississauga, ON, Canada) and a DNA template, pHCV1/SF919 (National Center for Biotechnology Information (NCBI) database accession number AF271632). The two primers used for the amplification were 1A5BH5 (5′-GCT AGG GCT AGC CAC CAC CAC CAC CAC CAC TCA ATG TCT TAC TCT TGG AC (SEQ ID NO: 504)) and 1A5BR4 (5′-CTC GAC CTC GAG TCA GCG GGG CCG GGC ATG AGA CAC (SEQ ID NO: 505)). Primer 1A5BH5 contains one Nhe I site followed by a series of six codons encoding a hexahistidine tag and the first amino acid of HCV NS5B polymerase from HCV genotype 1a. Primer 1A5BR4 contains one Xho I restriction site and sequence complementary to the last 21 nucleotides of the genotype 1a NS5B protein lacking the 21 amino acids at the C-terminal end. The PCR product obtained was first cloned into the intermediate vector pGEM-T (Promega Corp., Madison, Wis., USA). One clone was completely sequenced. A restriction fragment (Nhe I-Xho I) of 1737 base pairs (bp) was then sub-cloned into the expression vector pET-24d (Novagen Corp., Madison, Wis., USA) predigested with the identical set of restriction enzymes to generate an expression construct encoding HCV NS5B polymerase. pET-21b (Novagen Corp., Madison, Wis., USA) also is a suitable parent plasmid for constructing NS5B polymerase expression constructs.

### Example 3

This example describes the generation of an expression vector of the invention and a cell line of the invention for producing a polypeptide of the invention and HCV replicon cells. Methods for screening HCV NS5B polymerase protein and mutant HCV also are described.

### Plasmid Construction and Production of Recombinant Polymerase

All mutations were introduced into the pFKI389/NS3-3′/adapt vector (Krieger et al., J. Virol., 75, 4614-4624 (2001); Lohmann et al., J. Virol., 77, 3007-3019 (2003)) by PCR-based site-directed mutagenesis and standard recombinant DNA technologies. The pFKI389/NS3-3′/adapt vector is a “cell culture adapted HCV replicon” containing a R465G mutation within the NS5B gene, and was obtained from Reblikon GmbH, Gau-Odernheim, Germany. The “cell culture adapted replicon” was chosen instead of the wild-type replicon because the R465G mutation was found in all of the progeny RNA isolated from 9.13 replicon cells used in drug susceptibility studies to compare the fold shift in IC50s (described further below). The adaptive R465G mutation in the NS5B region was previously reported (Krieger et al., supra) and found to be very efficient in generating replicon cell colonies after RNA transfection into Huh-7 cells.

For in vitro fitness studies, mutations were also introduced into the pFKI389Luc/NS3-3′/5.1 vector, which contains the firefly luciferase gene instead of neo-gene and harbors the cell culture adaptive mutations of clone NK5.1 (Krieger et al., supra; Lohmann et al., supra). The integrity of all constructs was confirmed by double-stranded DNA sequencing

Linearized plasmids with Sca I and Ase I restriction enzymes were utilized to generate in vitro transcripts using the T7 MEGAScript Kit® and purified with the MEGAclear™ purification kit (Ambion, Austin, Tex., USA). RNA concentration was determined by measuring the optical density at 260 nm, and the RNA integrity was verified by subjecting the nucleic acid materials to denaturing agarose gel electrophoresis.

### Cell Lines and Stable HCV Genotype 1b Replicon Cells

The hepatocarcinoma Huh-7 cell line, the HCV replicon cell line Huh-7 9.13 (9.13 replicon cells), and the Huh-7-ET replicon cell line were obtained from Reblikon GmbH, Gau-Odernheim, Germany. Briefly, 9.13 replicon cells are derived from stably transfected Huh-7 cells with the HCV sub-genomic replicon 1377/N53-3′/wt (Lohmann et al., Science, 285, 110-113 (1995)). The Huh-7-ET cell line contains the cell culture-adapted replicon pFKI389luc-ubi-neo/NS3-3′/5.1 construct that carries, in addition to the neomycin gene, an integrated copy of the firefly luciferase gene (Vrolijk et al., J. Virol. Methods, 110(2), 201-209 (2003)). A Huh-7 sub-cell line (“ET-cured” cells) was established by “curing” the Huh-7-ET cell line containing the subgenomic replicon pPK 1389luc-ubi-neo/NS3-3′/5.1 by prolonged treatment (three weeks) with the human interferon alpha 2a without geneticin (G418) selection. The lack of residual replicon RNA in these cells was confirmed by RT-PCR and by cell-death upon treatment with low concentrations of G418.

### Stable HCV Genotype 1a Replicon Cells

A selectable subgenomic replicon of the HCV genotype 1a (named 1a-Neo-1a) was constructed using a three-step cloning procedure. Briefly, the IRES region from genotype 1a was amplified by PCR following cDNA synthesis using RNA extracted from a commercial-source of genotype 1a-infected serum. Using the diluted cDNA, a PCR product of approximately 320 bp was amplified using oligonucleotides 1a-IRES-H2 (5′-CAG CAT AAG CTT CGT AAT ACG ACT CAC TAT AGC CAG CCC CCT GA (SEQ ID NO: 506)) and 5′-NCR-R12 (5′-CAC TCG CAA GCA CCC TAT CA (SEQ ID NO: 507)). The product was cloned into pFK I377/NS3-3′/wt(19) to replace the IRES of genotype 1b with the IRES from genotype 1a. The resulting plasmid, named replicon 1a-Neo-1b, was used in the final cloning step in which the complete non-structural protein sequence and 3′ non-translated region from genotype 1b were replaced with those from genotype 1a. The complete fragment encompassing the non structural proteins (NS3 to the NS5B) from the Kpn I restriction site (located within the ECMV IRES) to the Spe I restriction site (located at the far 3′ end of the non-coding region) was synthesized commercially (Integrated DNA Technologies, Inc., Coralville, Iowa, USA). This sequence was based on the H77 sequence (GenBank NC_{—}004102 (Kolykhalov et al., J. Virol., 74(4), 2046-2051 (2000)) with minor sequence modifications, including the adaptive amino acid changes P1496L and S2204I (amino acids numbers refer to the location within the H77 full-length HCV genome (Yi et al., Proc. Natl. Acad. Sci., 103, 2310-5 (2006)). The final cloning step leading to replicon 1a-Neo-1a comprised cloning the Kpn I-Spe I from the synthetically synthesized DNA to replace the equivalent sequence in replicon 1a-Neo-1b.

RNA for replicon 1a-Neo-1a was synthesized with T7 MEGAScript reagents (Ambion, Austin, Tex., USA) after linearizing the corresponding replicon plasmid with Hpa I. Following treatment with RNase-free DNase to remove template DNA and purification with MEGAclear kit (Ambion), the RNA (0.01 to 1 μg) was transfected into the ET-cured cells by electroporation. The transfected cells were plated in T-75 flasks for selection of G418-resistant colonies. The medium was replaced with DMEM-10% FBS supplemented with 500 μg G418/mL 48 h post transfection. Media was then replaced every four days. Three weeks later, well isolated colonies were isolated and expanded for further analysis by real time PCR analysis. One stably transfected clone with replicon 1a-Neo-1a, named clone W11.8, was chosen and progeny RNA was analyzed and sequenced to confirm that no modifications were present within the polymerase sequence.

All cell lines were maintained in cultures at a sub-confluent level (<85%) as the level of replicon RNA is higher in actively proliferating cells. Cell lines were maintained in Dulbecco's modified Eagle minimal essential medium (DMEM) supplemented with 10% fetal bovine serum, 100 IU/mL of penicillin, 100 μg/mL streptomycin, non-essential amino acids, 2 mM glutamine, and 10 mM HEPES (Wisent Inc., St-Bruno, QC, Canada). Cells were incubated at 37° C., in an atmosphere of 5% CO₂ and passaged twice a week to maintain sub-confluence. The 9.13 replicon and other recombinant replicon cells were maintained in media containing 600 μg/mL of G418 (Invitrogen).

Stable replicon cells were generated using T7 transcripts derived from the individual recombinant plasmids as described (Lohmann et al., Science, 285, 110-113 (1999)). Following selection with G418 and expansion of selected colonies, total RNA was extracted and used to generate cDNAs. Amplification by PCR was then performed to amplify the region of interest, and mutations were confirmed by sequencing. One to three independent recombinant replicon stable cell lines were used for the IC50 determination of VX-222 and compared side by side with the wild-type parental 9.13 replicon cells.

### HCV Replicon Cell Assay to Determine Sensitivity to Agents Using Real-Time PCR

The 9.13 replicon cells and the stable recombinant replicon cells were seeded in a 12-well culture dish at a density of 3×10⁴ cells/well in a volume of 1 mL. The cell culture media used for the assay was DMEM supplemented with 10% fetal bovine serum, 100 IU/mL of penicillin, 100 μg/mL streptomycin, non-essential amino acids, 2 mM glutamine and 10 mM HEPES. After incubating for 3-4 hours, candidate agents (drugs) were added at various concentrations for a final volume of 2 mL of the same culture media. Cells were incubated for 4 days at 37° C. with 5% CO₂. Thereafter, total RNA (cellular and viral origin) was extracted using the RNeasy kit (Qiagen Inc.) according to the manufacturer's protocol. cDNA synthesis was performed using the MMLV reverse transcriptase and random hexamer primer (Invitrogen). The inhibitory effect of agents against wild-type replicon cells, resistant colonies, or recombinant replicon cells was determined by monitoring the levels of HCV RNA, normalized to cellular 18S ribosomal RNA, by real time PCR using the ABI PRISM® 7700 Sequence Detection System (Applied Biosystems, Foster City, Calif., USA).

The 50% inhibitory concentrations (IC50s) for agents were determined from dose response curves using six to ten concentrations per agent in triplicate. Curves were fitted to data points using nonlinear regression analysis, and IC50s were interpolated from the resulting curve using GraphPad Prism software, version 2.0 (GraphPad Software Inc., San Diego, Calif., USA).

### Replication Capacity of HCV Recombinant Variants

For electroporation, single-cell suspensions of “ET-cured cells” were prepared, washed once with phosphate-buffered saline, counted, and resuspended at a concentration of 10⁷ cells/mL in Cytomix transfection buffer (Lohmann et al., supra). Transcripts (10-20 μg) were mixed with 400 μL of cell suspension by pipetting, electroporated, and transfected cells were immediately resuspended in 40 mL complete DMEM culture medium as described above except that G418 or phenol red was not present. In this methodology, cells were subjected to electroporation using a Gene Pulser System (Bio-Rad, Mississauga, ON, Canada) in a cuvette with a gap width of 0.4 cm (Bio-Rad) under conditions applying 960 μF and 270 volts. Resuspended cells were seeded in white opaque 96-well microtiter plates (100 μL cell suspension per well out of 40 mL). Cells were then further incubated for a time period of four hours to four days at 37° C. in a 5% CO₂ incubator. For each time point post-transfection, the culture media was removed and cells were lysed by the addition of 95 μL of luciferase buffer (luciferin substrate in buffered detergent). Thereafter, cell lysates were incubated at room temperature, protected from direct light, for at least 10 minutes. Plates were read for luciferase counts using a luminometer (Perkin Elmer, MA, USA). At least two measurements were performed for all luciferase assays. Replication capacity of all recombinant variants were determined as the ratio of the luciferase signal at four days post-transfection divided by the luciferase signal at four hours post-transfection to normalize for the transfection efficiency. The replication capacity (fitness) of the HCV replicon mutants was expressed as their normalized replication efficiency compared to that of the wild type which was set at 100%.

### Selection and Characterization of Drug-Resistant HCV Replicon Progeny

Drug-resistant HCV replicons were selected as previously described. (See, e.g., Amparo et al., 46th Conference on Antimicrobial Agents and Chemotherapy (ICAAC). 2006, Abstract 1273; Le Pogam et al., J. Virol., 80(12), 6146-54 (2006); Lin et al., J. Biol. Chem., 279(17), 17508-17514 (2004); Lu et al., Antimicrob. Agents Chemother., 48(6), 2260-2266 (2004); Trozzi et al., J. Virol., 77, 3669-3679 (2003)). Briefly, approximately 2.5×10⁴ genotype 1a replicon cells (W11.8 replicon cells at passage 13) or genotype 1b replicon cells (9.13 replicon cells at passage greater than 100) were plated in 75 cm² cell culture flasks (Falcon 353136) in complete culture medium with high concentration of G418 (600 μg/mL). Once the cells were fully adhered, the culture medium was supplemented with various concentrations of VX-222 (ranging from 7 to 220-fold the IC50 value in replicon cells as evaluated by real time PCR). In addition, untreated cells were maintained in culture during the course of the experiment as negative control.

Media and drugs were replaced twice a week over the course of the experiment. Three to four weeks following the initial plating, resistant clones of cells (colonies) were counted and randomly picked for culture or for direct RNA extraction. In most cases, cells were expanded for a few passages in the presence of G418 but without VX-222. In other cases, colonies were directly used for the RNA extraction using a glass cloning cylinder (Bel-Art Products, Pequannock, N.J., USA).

Following random selection of colonies and cell growth, total RNA was extracted from individual clones using the RNeasy kit according to the manufacturer's protocol (Qiagen Inc., Mississauga, ON, Canada). Total cellular RNA (1-2 μg) from treated or untreated replicon cells was subjected to reverse transcription (RT) using the Moloney Murine Leukemia Virus (MMLV) RT enzyme and random primers (Invitrogen). This step was followed by PCR amplification of the C-terminal NS5B coding region using the appropriate pairs of oligonucleotides described in Table 1.

TABLE 1

| Name | 5′-Position | Polarity | Sequence (5′ to 3′) |
|---|---|---|---|
| 5B-H21 | 6493 ^{a} | + | GTT CGT GTG TGC GAG AAA ATG |
| | | | (SEQ ID NO: 508) |
| 3′-NCR-R9 | 7808 ^{a} | - | GAG GAT GGC CTA TTG GCC TGG A |
| | | | (SEQ ID NO: 509) |
| 5B-H9 ^{c} | 6733 ^{a} | + | CCC GAA GCC AGA CAG GCC ATA |
| | | | (SEQ ID NO: 510) |
| 1a-5BH2 | 8098 ^{b} | + | CGT GCG CGT GTG CGA GAA GAT G |
| | | | (SEQ ID NO: 511) |
| 1a-3′-NCR-R2 | 9403 ^{b} | - | AAG AGG CCG GAG TGT TTA CCC CAA C |
| | | | (SEQ ID NO: 512) |
| 1a-5B-R1 | 9376 ^{b} | - | TCA TCG GTT GGG GAG GAG GTA GAT |
| | | | (SEQ ID NO: 513) |

^{a} Relative to HCV subgenomic replicon I377/NS3-3′/wt
^{b} Relative to HCV H77 full-length
^{c} Oligonucleotides used for sequencing the PCR products

The C-terminal two-thirds of the NS5B coding sequence was amplified using 1a-5BH2 and 1a-3′-NCR-R2 as specific primers for the genotype 1a replicon RNA (PCR product size of 1306 bp). The PCR products were sequenced using 1a-5B-R1 sequencing primer. For the genotype 1b replicon RNA, the 5B-H21 and 3′NCR-R9 specific primers were used (PCR product size of 1316 bp). The resulting PCR products were sequenced using 5B-H9 sequencing primer. This strategy allowed complete sequencing of the region spanning from amino acids 262 to 578 for genotype 1a and from amino acids 259 to 591 for genotype 1b, which largely covers the thiophen binding-pocket.

The high-fidelity DNA polymerase Phusion Hot Start was used to amplify the cDNA according to the manufacturer's protocol (Finnzymes Oy, Epsoo, Finland). PCR conditions were as followed: each reaction took place in a final volume of 50 μl containing 1× high-fidelity buffer, 2 mM MgCl₂, 200 μM each dNTP, 0.5 μM of each primer, 3% DMSO, 1 unit of Phusion Hot Start DNA polymerase, and 1-2 μl of cDNA. A standard reaction condition was used for the great majority of all PCRs: an initial step at 98° C. for 2 min, followed with cycling for 38 to 40 cycles with 98° C. for 30 sec, 55° C. for 20 sec, 72° C. for 1 min, and a final elongation step at 72° C. for 10 min. Sequencing of the RT-PCR products, using specific primers located within the NS5B region, was carried out on an Applied Biosystems ABI 310 sequencer (McGill University and Génome Québec Innovation Centre, Montréal, QC, Canada).

### Results

Mutant replicons resistant to specific inhibitors were made using replicon constructs expressing the neomycin phosphotransferase gene (NPT). The method capitalized on two features of replicons. First, the low fidelity of replicon replication in tissue culture causes the accumulation of mutations, which leads to a degree of genetic diversity sufficient to mimic the complexity of the HCV quasispecies in infected patients. Second, the survival of replicon cells in the presence of G418 is dependent on replicon-driven expression of NPT. Cells containing mutant replicons with decreased sensitivity to an inhibitor give rise to colonies that can be counted, isolated, expanded and characterized.

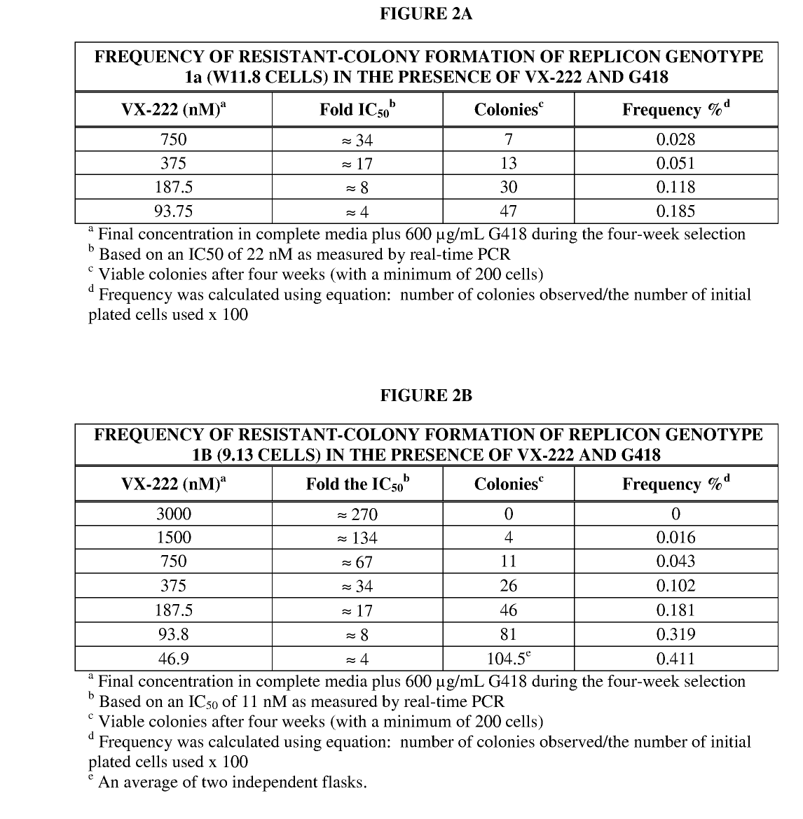

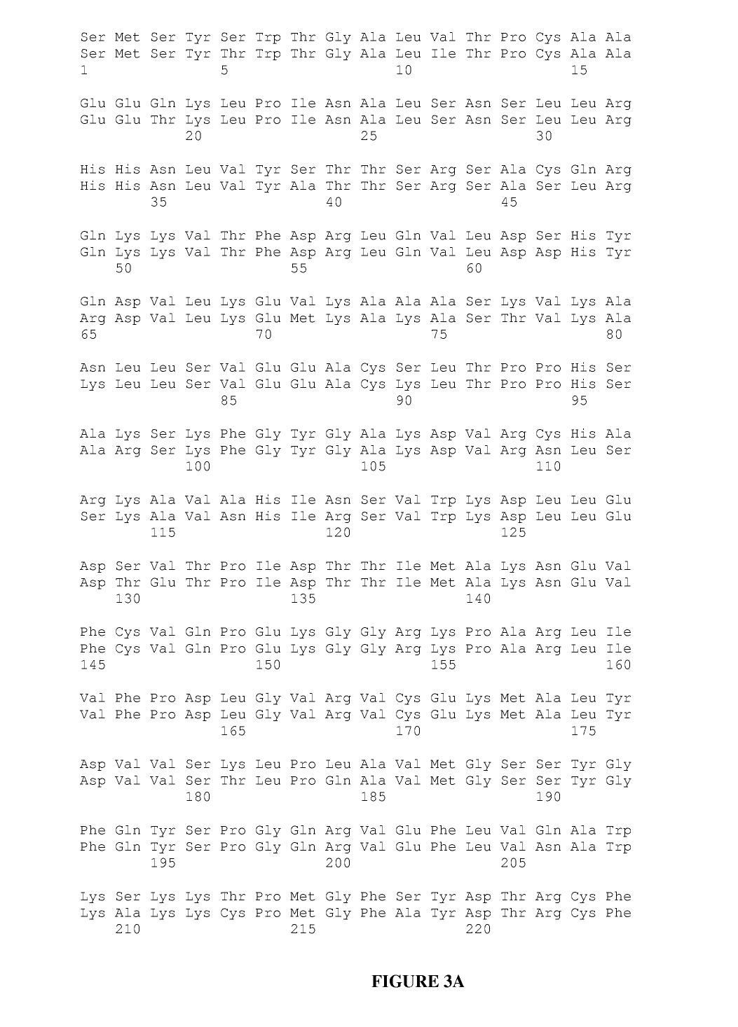

The results of two typical experiments for the selection of resistant colonies using both replicon cells of genotype 1a and 1b are provided in the tables in FIGS. 2A and 2B. The higher the concentration of VX-222 used for selection, the lower the number of surviving colonies, indicating that mutations conferring resistance were favored in the progeny HCV as a result of exposure to inhibitors. No colony could be readily detected for replicons of genotype 1b exposed to a VX-222 concentration of about 300 fold of the IC50, indicating that no mutants could be selected when VX-222 concentration exceeded a certain value (e.g., 300 fold the IC50 for genotype 1b).

The frequency of resistant clones at an equivalent IC50-fold concentration of VX-222 was higher for the genotype 1b replicon cell line than for the genotype 1a replicon cell line. However, the two cell lines each contain a different genotype replicon and are two independent cell clones; the experiments were not performed side by side; and the cell lines were not equally passaged and the replicon RNA population (quasispecies) diversity could be higher in replicon cells that have been grown for hundred of passages (replicon 1b).

Following selection of VX-222 resistant colonies, many independent resistant clones were randomly picked for each replicon cell line, and their progeny HCV RNA was analyzed genotypically by sequencing the C-terminal two-thirds of the NS5B gene. Amino acids residues M423, I482, and A486 were consistently found to mutate in both genotypes. All selected clones contained a mutation at position L419, M423, I482, or A486, except for one HCV variant having only a mutation at position A494. There was no apparent relationship between the drug concentration used and the mutation profile of these clones. For instance, in genotype 1b, M419, L482, and A486 mutations were randomly spread over the entire range of VX-222 concentrations, despite the fact that the mutations confer different degrees of resistance to VX-222 (see Table 2 below). Additionally, the resistance profile between the genotypes appears to differ; M423 and L482 were the most prevalent mutations in genotype 1a (63% of the 36 clones) while L419 and A486 were the most prevalent mutations in genotype 1b (74% of the 31 clones). The difference in resistance pattern could be due to the difference in the initial progeny diversity between genotypes.

TABLE 2
SENSITIVITY OF HCV REPLICON VARIANTS TO VX-222

| Replicon | IC₅₀ | IC₅₀ Fold-Change vs |
|---|---|---|
| (genotype) | (nM) | Wild-Type Replicon |
| L419M | 900 | ≈80 |
| L419S | >2,000 | >66 |
| M423T | 319 | ≈29 |
| M423V | 110 | ≈10 |
| M423I | 77.2 | ≈7.1 |
| I482L | 290 | ≈26 |
| A486V | 843 | ≈50 |
| V494A | 132 | ≈5.9 |

The role of amino acids L419, M423, I482, A486, and V494A in conferring resistance to VX-222 was investigated. A total of seven recombinant replicons with single mutations—L419M, M423V, M423T, M423I, I482L A486V, and V494A—were generated and stable replicon cell lines were obtained for each mutation. In addition, an original resistant colony with L419S genotype was employed to determine the susceptibility to VX-222. The stability of the mutations in the replicon cell lines was confirmed prior to their use by direct sequencing of the RT-PCR products using RNA extracted from these cell lines.

The activity of VX-222 against the wild-type replicon cells was compared to the recombinant mutant replicons (Table 2). An approximate 6- to 80-fold increase of VX-222's IC50 was recorded for all seven recombinant replicons, confirming the mutations' roles in VX-222 resistance. The M423V and M423I mutations were shown to confer about the same level of resistance to VX-222, 10 and 7.1-fold increase in IC50, respectively. The L419M, M423T, and A486V mutations conferred a higher level of resistance to VX-222 with 80, 29, and 50-fold increase of IC50, respectively. A greater than 66-fold increase in VX-222's IC50 was recorded with the L419S cell line.

This example describes the production of recombinant HCV NSSB polymerase and identification of mutations within the polymerase amino acid sequence conferring resistance to a polymerase inhibitor (VX-222). The assays described herein can be repeated with test agents (in addition to VX-222 or as a replacement for VX-222) to identify new agents that are effective (alone or in combination with VX-222) for inhibiting replication of HCV, including mutant HCV, such as the mutant HCV described herein.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. An isolated polypeptide comprising an amino acid sequence comprising at least one variation from SEQ ID NO: 1, the at least one variation selected from the group consisting of
cysteine, isoleucine, valine, or proline at amino acid position 419;
alanine, valine, or asparagine at amino acid position 482;
valine, isoleucine, threonine, or serine at amino acid position 486; and
isoleucine at amino acid position 494,
as the amino acid positions are defined in SEQ ID NO: 1, wherein the polypeptide has Hepatitis C Virus (HCV) NS5B polymerase activity.

2.-3. (canceled)

4. The isolated polypeptide of claim 1 comprising an amino acid sequence at least 90% identical to SEQ ID NO: 1.

5.-7. (canceled)

8. The isolated polypeptide of claim 1, wherein the amino acid residue of the polypeptide corresponding to amino acid 419 of SEQ ID NO: 1 is cysteine, isoleucine, valine, or proline.

9. The isolated polypeptide of claim 1, wherein the amino acid residue of the polypeptide corresponding to amino acid 482 of SEQ ID NO: 1 is alanine, valine, or asparagine.

10. The isolated polypeptide of claim 1, wherein the amino acid residue of the polypeptide corresponding to amino acid 486 of SEQ ID NO: 1 is valine, isoleucine, threonine, or serine.

11. The isolated polypeptide of claim 1, wherein the amino acid residue of the polypeptide corresponding to amino acid 494 of SEQ ID NO: 1 is isoleucine.

12.-20. (canceled)

21. An isolated polypeptide comprising an amino acid sequence at least 90% identical to the amino acid sequence of SEQ ID NO: 516 or at least 90% identical to a portion of SEQ ID NO: 516 having Hepatitis C Virus (HCV) NS5B polymerase activity, with the proviso that at least one of the amino acids of the polypeptide that correspond to positions 419, 482, 486, and/or 494 of SEQ ID NO: 516 is identical to the corresponding amino acid(s) of SEQ ID NO: 516.

22. The isolated polypeptide of claim 21, wherein the amino acid residue of the polypeptide corresponding to amino acid 419 of SEQ ID NO: 3 is cysteine, isoleucine, valine, or proline.

23. The isolated polypeptide of claim 21, wherein the amino acid residue of the polypeptide corresponding to amino acid 482 of SEQ ID NO: 3 is alanine, threonine, valine, or asparagine.

24. The isolated polypeptide of claim 21, wherein the amino acid residue of the polypeptide corresponding to amino acid 486 of SEQ ID NO: 3 is valine, isoleucine, threonine, or serine.

25. The isolated polypeptide of claim 21, wherein the amino acid residue of the polypeptide corresponding to amino acid 494 of SEQ ID NO: 3 is isoleucine.

26.-35. (canceled)

36. The isolated polypeptide of claim 1 fused to a heterologous peptide.

37. (canceled)

38. An isolated peptide having no more than 50 amino acids and comprising a sequence of 6-50 amino acids that is at least 90% identical to a portion of the amino acid sequence of SEQ ID NO: 516 encompassing at least one of amino acid residues 419, 482, 486, and 494, with the proviso that, when present, at least one of the amino acids of the polypeptide that corresponds to positions 419, 482, 486, and/or 494 of SEQ ID NO: 516 is identical to the corresponding amino acid(s) of SEQ ID NO: 516.

39.-41. (canceled)

42. An isolated polypeptide comprising at least one of the amino acid sequences selected from the group consisting of SEQ ID NOs: 4-502.

43. An isolated antibody or fragment thereof that specifically binds the polypeptide of claim 1.

44.-47. (canceled)

48. An isolated polynucleotide comprising a nucleotide sequence encoding the polypeptide of claim 1.

49.-53. (canceled)

54. An isolated polynucleotide comprising a nucleotide sequence that encodes at least one of the amino acid sequences selected from the group consisting of SEQ ID NOs: 4-502.

55.-59. (canceled)

60. An expression vector comprising the polynucleotide of claim 54.

61.-63. (canceled)

64. An isolated cell comprising the expression vector of claim 60.

65. (canceled)

66. An isolated cell transformed or transfected with a polynucleotide according to claim 48, wherein the cell expresses a polypeptide encoding by the polynucleotide, and wherein the cell is free of HCV infection.

67.-68. (canceled)

69. An assay mixture comprising an isolated polypeptide according to claim 1, an isolated RNA template, an RNA primer, nucleotide triphosphates, and buffer.

70. A method for characterizing the HCV inhibitory activity of an agent, the method comprising performing an HCV NS5B polymerase reaction with the polypeptide of claim 1 in the presence of an agent, and comparing polymerase activity in the presence of the agent with polymerase activity of the polypeptide in the absence of the agent.

71.-72. (canceled)

73. A method for detecting the presence of drug-resistant HCV in a sample comprising HCV, wherein the method comprises determining the presence or absence of an HCV NS5B polypeptide with an amino acid sequence in which at least one amino acid that corresponds to positions 419, 482, 486, and/or 494 of SEQ ID NO: 516 is identical to the corresponding amino acid(s) of SEQ ID NO: 516, wherein the presence of the at least one amino acid in the HCV NS5B protein indicates the presence of drug-resistant HCV.

74. The method of claim 73, wherein determining the presence or absence of the polypeptide comprises contacting the sample with an antibody or fragment thereof that specifically binds said polypeptide and detecting binding of the antibody or fragment thereof to the polypeptide.

75. A method for detecting the presence of drug-resistant HCV in a sample, wherein the method comprises determining the presence or absence of a polynucleotide in the sample, the polynucleotide comprising a nucleic acid sequence encoding HCV NS5B polymerase containing at least one codon selected from the group consisting of
a codon encoding cysteine, isoleucine, valine, or proline at a position corresponding to codon position 419 of SEQ ID NO: 2;
a codon encoding alanine, valine, or asparagine at a position corresponding to codon position 482 of SEQ ID NO: 2;
a codon encoding valine, isoleucine, or serine at a position corresponding to codon position 486 of SEQ ID NO: 2; and
a codon encoding isoleucine at a position corresponding to codon position 494 of SEQ ID NO: 2,
wherein the presence of the at least one codon indicates the presence of drug-resistant HCV in the sample.

76. The method of claim 75, wherein the method comprises
contacting polynucleotides in the sample with a nucleic acid probe, under conditions allowing sequence-specific hybridization of the nucleic acid probe with a target sequence, wherein hybridization of the nucleic acid probe to a polynucleotide in the sample indicates the presence of drug-resistant HCV in the sample, wherein the nucleic acid probe comprises nucleotide sequence of 14-50 nucleotides complementary to a continuous portion of a nucleotide sequence of a of nucleotide encoding a polypeptide having HCV NS5B polymerase activity, the nucleotide having at least one codon variation of SEQ ID NO: 2 selected from the group consisting of a codon encoding cysteine, isoleucine, valine, or proline at codon 419; a codon encoding alanine, valine, or asparagine at codon 482; a codon encoding valine, isoleucine, threonine, or serine at codon 486; and a codon encoding isoleucine at codon 494, as the codon positions are defined in SEQ ID NO: 2, the portion including at least one codon at a codon position selected from codon positions 419, 482, 486, and 494 of SEQ ID NO: 2.

77. (canceled)

78. A method for determining whether an HCV-infected patient is infected with an HCV strain that has a decreased sensitivity to VX-222, the method comprising determining the presence or absence of a polypeptide in a biological sample from the patient, the polypeptide comprising an amino acid sequence comprising at least one variation from SEQ ID NO: 1, the at least one variation selected from the group consisting of
cysteine, isoleucine, methionine, serine, valine, or proline at amino acid position 419;
lysine at amino acid position 422;
alanine, isoleucine, threonine, or valine at amino acid position 423;
alanine, leucine, threonine, valine, or asparagine at amino acid position 482;
valine, isoleucine, threonine, or serine at amino acid position 486; and
isoleucine or alanine at amino acid position 494,
as the amino acid positions are defined in SEQ ID NO: 1, wherein the presence of the polypeptide indicates infection with an HCV strain that has a decreased sensitivity to VX-222.

79. The method of claim 78, wherein determining the presence or absence of the polypeptide comprises contacting the sample with an antibody or fragment thereof that specifically binds said polypeptide and detecting binding of the antibody or fragment thereof to the polypeptide.

80. A method for identifying an agent able to rescue the polymerase-inhibitor activity of VX-222 against an HCV NS5B polymerase having resistance to VX-222, the method comprising:
a) performing an HCV NS5B polymerase reaction with a polypeptide comprising an amino acid sequence comprising at least one variation from SEQ ID NO: 1, the at least one variation selected from the group consisting of
cysteine, isoleucine, methionine, serine, valine, or proline at amino acid position 419;
lysine at amino acid position 422;
alanine, isoleucine, threonine, or valine at amino acid position 423;
alanine, leucine, threonine, valine, or asparagine at amino acid position 482;
valine, isoleucine, threonine, or serine at amino acid position 486; and
isoleucine or alanine at amino acid position 494,
as the amino acid positions are defined in SEQ ID NO: 1, in the presence of an agent and VX-222; and
b) comparing polymerase activity of the polypeptide in the presence of the agent with polymerase activity of the polypeptide in the absence of the agent, wherein a decrease in HCV polymerase activity in the presence of the agent is indicative of the ability to rescue the polymerase-inhibitory activity of VX-222 against an HCV NSSB polymerase having resistance to VX-222.

81. (canceled)

82. A method for determining whether an HCV-infected patient is infected with an HCV strain that has a decreased sensitivity to VX-222, the method comprising determining the presence or absence of a polynucleotide in a biological sample from the patient, the polynucleotide comprising a nucleic acid sequence encoding HCV NS5B polymerase containing at least one codon selected from the group consisting of a codon encoding cysteine, isoleucine, methionine, serine, valine, or proline at a position corresponding to codon position 419 of SEQ ID NO: 2;
a codon encoding lysine at a position corresponding to codon position 422 of SEQ ID NO: 2;
a codon encoding alanine, isoleucine, threonine, or valine at a position corresponding to codon position 423 of SEQ ID NO: 2;
a codon encoding alanine, leucine, threonine, valine, or asparagine at a position corresponding to codon position 482 of SEQ ID NO: 2;
a codon encoding valine, isoleucine, threonine, or serine at a position corresponding to codon position 486 of SEQ ID NO: 2; and
a codon encoding isoleucine or alanine at a position corresponding to codon position 494 of SEQ ID NO: 2,
wherein the presence of the polynucleotide indicates infection with an HCV strain having a decreased sensitivity to VX-222.

83. (canceled)
